(19) United States
(12) Patent Application Publication
(10) Pub. No.: US 2016/0199500 A1
(43) Pub. Date: Jul. 14, 2016

(54) COMPOSITIONS COMPRISING VITAMIN B12 AND INTRINSIC FACTOR AND METHODS OF USE THEREOF

(71) Applicant: Robert DOYLE, Manlius, NY (US)

(72) Inventor: Robert Doyle, Manlius, NY (US)

(73) Assignee: Syracuse University, Syracuse, NY (US)

(21) Appl. No.: 14/913,587

(22) PCT Filed: Aug. 22, 2014
(86) PCT No.: PCT/US14/52381

§ 371 Date: Feb. 22, 2016

Related U.S. Application Data
(60) Provisional application No. 61/868,841, filed on Aug. 22, 2013.

(51) Int. Cl. A61K 47/48; A61K 31/714; A61K 51/04

(52) U.S. Cl. CPC A61K 47/48107; A61K 47/48246; A61K 51/0472; A61K 51/0497; A61K 31/714

(57) ABSTRACT

A system for detecting cancer cells that targets the cubilin receptor for the vitamin B12 binding protein, intrinsic factor. A B12 conjugate and intrinsic factor is injected into the blood of a patient. The binding of intrinsic factor to cubilin will allow for receptor-mediated endocytosis and cellular internalization by cancerous cells, thereby allowing for detection via imaging or treatment.

### CROSS REFERENCE TO RELATED APPLICATIONS

This application claims the benefit of PCT Application PCT/US2014/052381, filed Aug. 22, 2014, which claims the benefit of U.S. provisional application No. 61/868,841, filed Aug. 22, 2013, each of the disclosures of which is hereby incorporated by reference in its entirety.

### FIELD OF THE INVENTION

The present invention relates to diagnostic and/or therapeutic compositions and methods comprising radiolabeled B₁₂ conjugates and intrinsic factor for non-oral administration.

### BACKGROUND OF THE INVENTION

According to the kidney cancer association, more than 1.3 million people were diagnosed with cancer in the United States in 2012. Of those diagnosed 50,000 had kidney cancer, with renal cell carcinoma (RCC) being the most common type. RCC has very few symptoms in the early stages so it often goes undetected until the tumor has grown large or tumor cells have metastasized to other parts of the body. Because 30-40% of RCC patients will develop metastasized tumors and patients with untreated metastasized tumors have a 5-year survival rate of up to 18 percent, early detection is of critical importance.

The site-specific drug targeting of tumor tissue holds great potential for enhanced treatment response, while also potentially reducing side effects. The B₁₂ uptake pathway has generated increasing interest over the past few years, due to its potential for specific drug delivery. B₁₂ is a non-toxic water-soluble vitamin that is vital for cell growth and proliferation, due to its role in DNA methylation and methionine production.

Previous diagnostic imaging techniques have used single photon emission computed tomography (SPECT) to track the enhanced B₁₂ demand in pigs, mice, dogs and humans bearing occult tumors. This was achieved using B₁₂ conjugates labeled with indium-111 and technectium-99m. PET imaging has recently been used to detect the cellular uptake of B₁₂-Cu⁶⁴ via TCII/CD320 endocytosis.

Cancer cells are characterized as cells that have a greater need for nutrients due to their over-proliferation. Among these nutrients used is vitamin B₁₂ (B₁₂). B₁₂ is absorbed during digestion and transported to the blood plasma via three binding proteins: haptocorrin (HC), transcobalamin II (TCII) and intrinsic factor (IF). Once in the blood B₁₂ is bound to either TCII or HC. The TCII receptor allows for the uptake of B₁₂ into cells.

B₁₂ labeled Cu⁶⁴ has been used to target the TCII receptor, CD320. However, because the TCII receptor is expressed throughout the body, high background imaging was observed. Therefore, there is a need for a new imaging technique that overcomes the issue of high background thereby allowing accurate detection of cancers.

### SUMMARY OF THE INVENTION

In an aspect, the present disclosure encompasses a pharmaceutical formulation for parenteral administration. The pharmaceutical formulation comprises intrinsic factor and B₁₂ or an analog thereof and a pharmaceutically acceptable carrier for parenteral administration, wherein the B₁₂ or analog thereof is conjugated to a detectable label and/or therapeutic agent.

In another aspect, the present disclosure encompasses a pharmaceutical formulation for intravenous administration. The pharmaceutical formulation comprises intrinsic factor and B₁₂ or an analog thereof and a pharmaceutically acceptable carrier for intravenous administration, wherein the B₁₂ or analog thereof is conjugated to a detectable label and/or therapeutic agent.

In still another aspect, the present disclosure encompasses a method of detecting a tumor in a subject. The method comprises administering to the subject a composition comprising intrinsic factor and B₁₂, wherein the B₁₂ is conjugated to a detectable label, and detecting the binding of the composition to cubilin in a subject, wherein the presence of the detectable label in a tissue that does not typically express cubilin indicates the presence of a tumor in the subject.

In yet still another aspect, the present disclosure encompasses a method of detecting a tumor in a subject. The method comprises administering to the subject a composition comprising intrinsic factor and B₁₂, wherein the B₁₂ is conjugated to a detectable label, and detecting the binding of the composition to cubilin in a subject, wherein the asymmetrical presence of the detectable label in a tissue comprising cells that are known to express cubilin indicates the presence of a tumor in the subject.

In a different aspect, the present disclosure encompasses a method of treating a tumor in a subject. The method comprises administering to the subject a composition comprising intrinsic factor and B₁₂, wherein the B₁₂ is conjugated to a therapeutic agent.

In other aspects, the present disclosure encompasses a method of delivering B₁₂ to a cell that expresses cubilin in a subject. The method comprises administering a complex of IF and B₁₂ to the subject intravenously.

In an additional aspect, the present disclosure encompasses a method of blocking cubilin function. The method comprises administering a complex of IF and B₁₂ to the subject intravenously.

### BRIEF DESCRIPTION OF THE FIGURES

The application file contains at least one drawing executed in color. Copies of this patent application publication with color drawing(s) will be provided by the Office upon request and payment of the necessary fee.

FIG. 1 depicts the structure of Vitamin B₁₂ showing the Co (III) atom coordinated by the four nitrogens of the corrin ring, dimethylbenzimidazole (DMB), and the X group which can be a cyanide, methyl, or deoxyadenosyl groups.

FIG. 2 depicts a schematic representing the B₁₂ dietary uptake pathway (HC=haptocorrin, IF=intrinsic factor, CB=cubilin, AM=amnionless, MRP1=multidrug resistant protein 1, MG=megalin).

FIG. 3 depicts a schematic representation of IF-B₁₂ probe synthesis.

FIG. 4 depicts a schematic of 1,1-bisthiazolate-(1,4)-diaminobutane reaction. i. NaBH(COOCH₃)₃ in anhydrous DCE, 16 h, under argon; ii. 10% TFA, 10% MeOH in water for 3h.

FIG. 5 depicts a RP-HPLC trace of 1 with a Tᵣ of 4.39 minutes.

FIG. 6 depicts a ¹H-NMR of 1 in D₂O on a 400 MHz NMR. ‡ is a MeOH impurity.

FIG. 7 depicts a RP-HPLC of 2 with a Tᵣ of 11.9 minutes.

FIG. 8 depicts a MALDI-ToF of 2 using a CHCA matrix spiked with 0.1% TFA (10 mg/mL CHCA). The expected m/z was 1640 m/z, and the m/z that was found was 1639.923.

FIG. 9 depicts a ¹H-NMR of 2 in D₂O run on a 400 MHz NMR machine. The characteristic 5 peaks for B₁₂ are present between 6-7.5 ppm.

FIG. 10 depicts a RP-HPLC of 2′ with a Tᵣ of 5.28 minutes.

FIG. 11 depicts a MALDI-ToF of 2′ using a CHCA matrix spiked with 0.1% TFA (10 mg/mL CHCA). The expected m/z was 1907 m/z, and the found m/z was 1907.615 m/z.

FIG. 12 depicts an emission profile of 2′ when excited at 488 nm with a λₘₐₓ of 566 nm. The slit width was 4 nm.

FIG. 13 depicts a schematic of an embodiment of a radiolabeled B₁₂ conjugate that may be pre-bound to Intrinsic Factor prior to administration to a subject.

FIG. 14 depicts the structure of L-propargyl glycine chlelated to metal. ‘M’ may be ^{99m}Tc.

FIG. 15 depicts the structure of a B₁₂ conjugate with a 5′-OH ribose attachment.

FIG. 16 depicts the structure of a B₁₂ conjugate with a b-position attachment.

FIG. 17 depicts the structure of a B₁₂ conjugate with a cobalt-C bond attachment.

FIG. 18A, FIG. 18B, FIG. 18C and FIG. 18D depict structures of various B₁₂ conjugates. (FIG. 18A) 1 is the structure of a B₁₂ conjugate modified at the 5′-OH of the ribose. (FIG. 18B) 2 is the structure of a B₁₂ conjugate modified at the cobalt ion. (FIG. 18C) 3 is the structure of a B₁₂ conjugate modified at the b-position. (FIG. 18D) 4 is the structure of a B₁₂ conjugate modified at the 5′-OH of the ribose.

FIG. 19A, FIG. 19B, FIG. 19C and FIG. 19D depict receptor mediated uptake of vitamin B₁₂-conjugate systems of >160 kDa. (FIG. 19A) Immunostaining with fluorescently-tagged anticubilin antibody. (FIG. 19B) Uptake of 1 tagged with CypHer 5E fluorsecent dye (1_{C5E}). (FIG. 19C) Colocalization of 1_{C5E} (red) and IF₄₀₅ (blue). Areas of colocalization are shown in purple. (FIG. 19D) Uptake of 2_{C5E} (red) and IF₄₀₅ (blue) in BeWo cells. Areas of colocalization are shown in purple.

### DETAILED DESCRIPTION OF THE INVENTION

The present invention comprises a composition for targeting cubilin expressing cells that uses a cubilin/IF mediated uptake system. Of note, the composition comprising IF and a B₁₂-conjugate is administered via the blood of a subject. Such a composition may be used to image and/or treat cancer cells. Prior to this disclosure, it was unknown that IF-B₁₂ could be administered via a non-oral route (i.e. intravenously) and effectively bind cubilin. In the B₁₂ dietary uptake pathway, when B₁₂ is released in the mouth from food, haptocorrin (HC) binds B₁₂ and carries it through the stomach into the small intestine. Due to the increase in pH and the presence of proteases, HC releases B₁₂ to be sequentially bound by intrinsic factor (IF). The IF-B₁₂ then travels through the small intestine until it binds to the cubilin receptor found on ileal enterocyte cells in the duodenum. At no point during the pathway of B₁₂ uptake is IF present in the blood; instead IF is present only in the gastrointestinal tract. As such, it was unexpected that IF-B₁₂ administered intravenously can locate and bind to cubilin outside of the gastrointestinal tract (i.e. non-ileal cubilin).

The inventors have found that a labelled B₁₂ probe pre-bound to IF, capable of binding to the cubilin receptor, will achieve both an increase in target specificity as well as a significant decrease in background emission. Targeting the cubilin receptor for site-specific uptake may increase specificity over previous B₁₂ receptor mediated uptake systems because cubilin is not expressed in all tissues, unlike transcobalamin II (TCII). For example, a B₁₂ probe not bound to IF achieves high background due to binding to TCII which is present throughout the body and is less specific.

### I. Composition

The present invention encompasses a composition comprising intrinsic factor and B₁₂ or an analog thereof, wherein the B₁₂ or analog thereof is conjugated to a detectable label and/or a therapeutic agent. B₁₂ analogs may be modified to improve bioavailability, solubility, stability, handling properties, or a combination thereof, as compared to an unmodified version. Thus, in another aspect, a composition of the invention comprises a modified B₁₂ or B₁₂ analog. In still another aspect, a composition of the invention comprises a prodrug of B₁₂ or a B₁₂ analog.

A composition of the invention may further comprise a pharmaceutically acceptable excipient, carrier or diluent. Further, a composition of the invention may contain preserving agents, solubilizing agents, stabilizing agents, salts (substances of the present invention may themselves be provided in the form of a pharmaceutically acceptable salt), buffers, or antioxidants.

### (a) Vitamin 8₁₂ (Cobalamin)

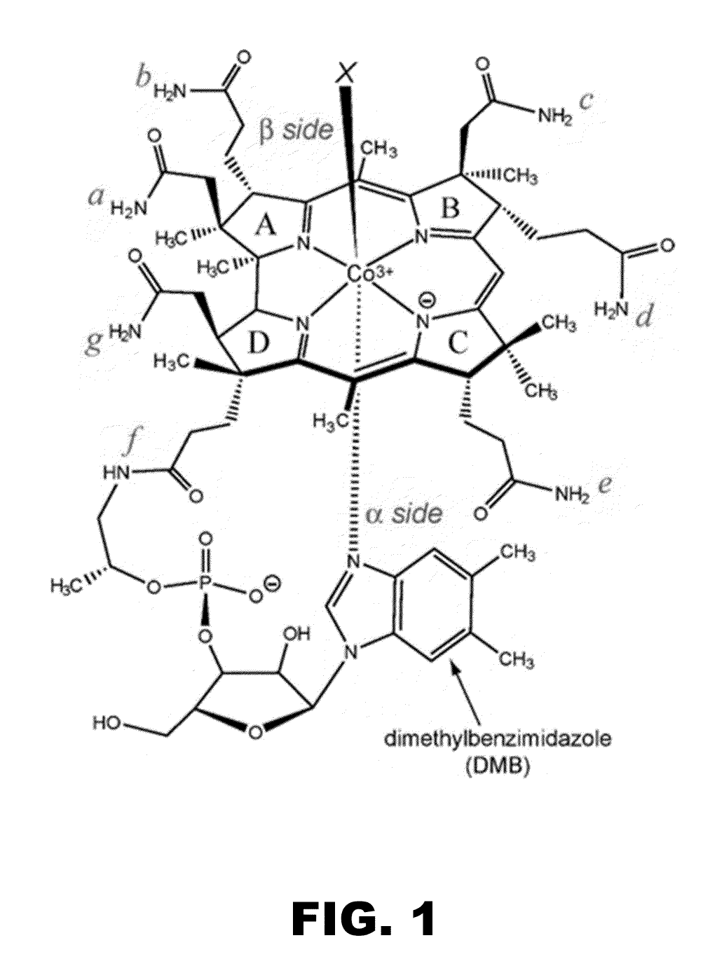

Vitamin B₁₂ is a water-soluble vitamin with a highly complex structure, comprising a midplanar corrin ring composed of four pyrroline elements linked to a central cobalt(III) atom. Throughout the disclosure vitamin B₁₂, B₁₂ and cobalamin may be used interchangeably and is depicted in FIG. 1.

In the structure of vitamin B₁₂, the central cobalt(III) atom is six-coordinated, with the equatorial positions filled by the nitrogen atoms of the corrin macrocycle. The (conventionally) ‘lower’, ‘α’-axial site is occupied by an imidazole nitrogen atom from a 5′,6′-dimethylbenzimidazole (DMB) base whereas the ‘upper’, ‘β’-axial site can be occupied by various X groups (e.g. CN⁻, CH₃⁻, Ado⁻, SCN⁻, SeCN⁻, SO₃⁻ and thiourea). The corrin ring incorporates seven amide side chains, three acetamides (a, c, g) and four propionamides (b, d, e, f). The four pyrrole rings are usually indicated as A, B, D and D, as shown in FIG. 1.

Several functional groups are readily available for modification on B₁₂, including propionamides, acetamides, hydroxyl groups, the cobalt(III) ion and the phosphate moiety. Accordingly, a B₁₂ conjugate of the invention may be modified at a propionamide, acetamide, hydroxyl group, the cobalt(III) ion and the phosphate moiety, provided the B₁₂ conjugate binds IF. Non-limiting examples of modification sites for a B₁₂ conjugate of the invention include at the a-position or b-position on the A-ring, at the c-position or d-position on the B-ring, at the e-position on the C-ring, at the g-position on the D ring, at the f-position, at the phosphate moiety, at the 5′- or 2′-hydroxyl on the ribose, and at the cobalt ion. Preferred sites of modification may include sites on the A ring such as the b-position, sites on the C ring such as the e-position, sites on the ribose unit such as the 5′-hydroxyl group, and the cobalt cation. Specifically, the e-position may be modified to allow interaction with IF. Alternatively, the b-position may be modified to disrupt TCII binding specifically. However, other sites of modification may be utilized provided they maintain the binding affinity of B₁₂ for IF. Preferable modifications may be those that maintain binding affinity for IF but reduce binding affinity for TCII. For example, the interactions between B₁₂ and TCII may be disrupted by utilizing the b-propionamide site after modification of the b-monocarboxylic acid. Such a modification may reduce the prevalence of TCII based uptake of the conjugates into healthy cells.

Methods for modification to B₁₂ are known in the art. The following provides non-limiting examples of methods for modification. It is contemplated that various other methods for modification common in the art of synthetic chemistry may be used. For example, carefully controlled partial hydrolysis of cyanocobalamin under acidic conditions gives access to desirable b and e acids. Methods for 5′-OH functionalization may rely on the reaction of cyanocobalamin ((CN)Cbl) with anhydrides, furnishing unstable ethers. Another method for conjugation may be the carbamate or carbonate methodology as described by Russell-Jones (WO 1999/065390, which is hereby incorporated by reference in its entirety). Briefly, the hydroxyl group at position 5′ is first reacted with a carbonyl group equivalent—1,1′-carbonyldiimidazole (CDl) or 1,1′-carbonylbis(1,2,4-triazole) (CDT)—and then treated with an amine or an alcohol giving carbamates and carbonates, respectively, at the 5′-position of the ribose tail. Alternatively, the 5′-OH group can be oxidized to the corresponding carboxylic acid using the 2-iodoxybenzoic acid (IBX)/2-hydroxypyridine (HYP) system as an oxidant and then coupled with amines. Another effective approach may rely on [1,3] dipolar cycloaddition. The 5′-OH is transformed into a good leaving group and subsequently substituted with an azide. The resulting “clickable” azide is stable and highly active in the copper-catalyzed as well as in the strain promoted [1,3] dipolar cycloaddition (CuAAC or SPAAC) to alkynes. This methodology is described in detail in Chrominski et al, Chem Eur J 2013; 19: 5141-5148, which is hereby incorporated by reference in its entirety. In a specific embodiment, the 5′-OH is transformed into an azide. An alkyne containing glycine is then added using “click” chemistry, which may then be chelated to a metal. In another specific embodiment, an alkyne comprising glycine is added at the b-position, which may be then be chelated to a metal. In still yet another specific embodiment, an alkyl chain linker may added be prior to the group responsible for metal chelation.

Functionalization of the cobalt ion may be accomplished by either alkylation or utilization of cyanide ligand properties to act as an electron pair donor for transition metals, resulting in bimetallic complexes. The synthesis of organometallic species requires reduction of the cobalt(III) to cobalt(I) B₁₂ and its subsequent reaction with electrophiles: alkyl halides, acyl halides, Michael acceptors, epoxides, etc. Alternatively, reduction may not be required and instead, the direct reaction of (CN)Cbl with terminal alkynes in the presence of Cu(I) salts may furnish acetylides in excellent yields. This methodology may allow the conjugation of two moieties to B₁₂ and is described in further detail in Chrominski et al, J Org Chem 2014; 79: 7532-7542, which is hereby incorporated by reference in its entirety. Accordingly, it is contemplated that two detectable labels and/or therapeutic agents may be conjugated to B₁₂. Briefly, using this methodology, “doubly clickable” vitamin B₁₂, a valuable building block for further functionalization via [1,3] dipolar azide-alkyne cycloaddition, may be prepared. A combination of AAC (CuAAC and SPAAC) with the carbamate method may allow conjugation at both the central cobalt ion and the 5′-position. In a specific embodiment, an alkyne comprising glycine may be added at the cobalt ion, which may then be chelated to a metal. In another specific embodiment, an alkyl chain linker may or may not be added prior to the group responsible for metal chelation.

B₁₂ or an analog thereof and a detectable label and/or therapeutic agent may be: i) conjugated directly together; ii) held apart by a ‘linker’ to produce distance between the B₁₂ or an analog thereof and the detectable label and/or therapeutic agent; or iii) conjugated to carriers that can couple the desired detectable label and/or therapeutic agent unconjugated, within the carrier. The detectable label and therapeutic agent are described in more detail in Section I(b) and Section I(c), respectively.

In an aspect, B₁₂ or an analog thereof may be conjugated to a detectable label and/or therapeutic agent directly via a covalent bond or indirectly via charge interaction. Non-limiting examples of a charge interaction may include ionic, hydrophobic, hydrogen bonding or Van der Waals forces. In an embodiment where B₁₂ or an analog thereof is coupled directly to a detectable label and/or therapeutic agent, a linker may or may not be used.

In another aspect, B₁₂ or an analog thereof may be conjugated to a carrier that can couple the desired detectable label and/or therapeutic agent unconjugated, within the carrier. Non-limiting examples of suitable carriers may include chelating agents. For example, B₁₂ or an analog thereof may be conjugated to a chelating agent that can couple the desired detectable label and/or therapeutic agent. The chelating agent may be directly conjugated to B₁₂ or an analog thereof or may be conjugated to a linker that is conjugated to B₁₂ or an analog thereof. As used herein, a “chelating agent” is a molecule that forms multiple chemical bonds with a single metal atom. Prior to forming the bonds, the chelating agent has more than one pair of unshared electrons. The bonds are formed by sharing pairs of electrons with the metal atom.

Examples of chelating agents include, but are not limited to, iminodicarboxylic and polyaminopolycarboxylic reactive groups, diethylenetriaminepentaacetic acid (DTPA), 1,4,7,10-tetraazacyclododecane-1,4,7,10-tetraacetic acid (DOTA), tetramethyl heptanedionate (TMHD), 2,4-pentanedione, ethylenediamine-tetraacetic acid disodium salt (EDTA), ethyleneglycol-O,O′-bis(2-aminoethyl)-N,N,N′,N′-tetraacetic acid (EGTA), N-(2-hydroxyethyl)ethylenediamine-N,N′,N′-triacetic acid trisodium salt (HEDTA), nitrilotriacetic acid (NTA), and 1,4,8,11-tetraazacyclotetradecane-N,N′,N″,N′″-tetraacetic acid (TETA), deferoxamine (DFO), 1,4,7-triazacyclononane-1,4,7-triacetic acid (NOTA), organic acids and amino acids such as citric acid, tartaric acid, gluconic acid and glycine, and derivatives thereof.

Chelating agents may be attached to B₁₂ or an analog thereof using methods generally known in the art. The following provides non-limiting examples of methods to attach chelating agents. It is contemplated that various other methods for attaching chelating agents common in the art of synthetic chemistry may be used. For example, B₁₂ or an analog thereof may be conjugated to a chelating agent by reacting a free amino group of B₁₂ or an analog thereof with an appropriate functional group of the chelator, such as a carboxyl group or activated ester. For example, B₁₂ or an analog thereof may be coupled to the chelator ethylenediaminetetraacetic acid (EDTA), common in the art of coordination chemistry, when functionalized with a carboxyl substituent on the ethylene chain. Synthesis of EDTA derivatives of this type are reported in Arya et al., (Bioconjugate Chemistry. 2:323, 1991), wherein the four coordinating carboxyl groups are each blocked with a t-butyl group while the carboxyl substituent on the ethylene chain is free to react with the amino group of B₁₂ or an analog thereof thereby forming a conjugate.

B₁₂ or an analog thereof may be coupled to a metal chelator component that is peptidic, i.e., compatible with solid-phase peptide synthesis. In this case, the chelator may be coupled to B₁₂ or an analog thereof in the same manner as EDTA described above.

B₁₂ or an analog thereof may be complexed, through its attached chelating agent, to a detectable label, thereby resulting in a B₁₂ or an analog thereof conjugate that is indirectly labeled. Similarly, cytotoxic or therapeutic agents may also be attached via a chelating group to B₁₂ or an analog thereof. As such, the chelating agent may be conjugated directly to the detectable label or therapeutic agent. Alternatively, an intervening amino acid sequence or linker can be used to conjugate the detectable label or therapeutic agent to the chelating agent.

In another aspect, B₁₂ or an analog thereof and the detectable label and/or therapeutic agent may be held apart by a linker to produce distance between the B₁₂ or an analog thereof and the detectable label and/or therapeutic agent. It is to be understood that conjugation of the B₁₂ or an analog thereof to the detectable label and/or therapeutic agent will not adversely affect either the binding function of the B₁₂ or an analog thereof to IF or the function of the detectable label and/or therapeutic agent. Suitable linkers include, but are not limited to, amino acid chains and alkyl chains functionalized with reactive groups for conjugating to both the B₁₂ or analog thereof and the detectable label and/or therapeutic agent.

In an embodiment, the linker may include amino acid side chains, referred to as a peptide linker. Accordingly, amino acid residues may be added to B₁₂ or an analog thereof for the purpose of providing a linker by which B₁₂ or an analog thereof can be conveniently affixed to a detectable label and/or therapeutic agent, or carrier. Amino acid residue linkers are usually at least one residue and can be 40 or more residues, more often 1 to 10 residues. Typical amino acid residues used for linking are tyrosine, cysteine, lysine, glutamic and aspartic acid, or the like.

In another embodiment, an alkyl chain linking group may be conjugated to B₁₂ or an analog thereof. For example, by reacting an amino group of B₁₂ or an analog thereof with a first functional group on the alkyl chain, such as a carboxyl group or an activated ester. Subsequently a chelator may be attached to the alkyl chain to complete the formation of a complex by reacting a second functional group on the alkyl chain with an appropriate group on the chelator. The second functional group on the alkyl chain is selected from substituents that are reactive with a functional group on the chelator while not being reactive with B₁₂ or an analog thereof. For example, when the chelator incorporates a functional group, such as a carboxyl group or an activated ester, the second functional group of the alkyl chain linking group can be an amino group. It will be appreciated that formation of the conjugate may require protection and deprotection of the functional groups present in order to avoid formation of undesired products. Protection and deprotection are accomplished using protecting groups, reagents, and protocols common in the art of organic synthesis. It will be appreciated that linking groups may alternatively be coupled first to the chelator and then to B₁₂ or an analog thereof. An alkyl chain linking group may be one to 40 or more carbons long, more often 1 to 10 carbons long. In a specific embodiment, an alkyl chain linking group may be 1, 2, 3, 4, 5, 6 or 7 carbons long. In another specific embodiment, an alkyl chain linking group may be 3 carbons long. In still another specific embodiment, an alkyl chain linking group may be 4 carbons long. In yet still another specific embodiment, an alkyl chain linking group may be 5 carbons long.

An alternative chemical linking group to an alkyl chain is polyethylene glycol (PEG), which is functionalized in the same manner as the alkyl chain described above for incorporation in the conjugates. B₁₂ or an analog thereof may be PEGylated for improved systemic half-life and reduced dosage frequency. In an embodiment, PEG may be added to a linker. As such, B₁₂ or an analog thereof may comprise a linker and PEG. For example, B₁₂ or an analog thereof may comprise an alkyl linker, one or more chelators and PEG.

### (b) Detectable Label

In an aspect, B₁₂ or an analog thereof may be conjugated to a detectable label. The detectable label may be directly conjugated to B₁₂ or an analog thereof or may be indirectly conjugated to B₁₂ or an analog thereof. In an embodiment, the detectable label may be complexed with a chelating agent that is conjugated to B₁₂ or an analog thereof. In another embodiment, the detectable label may be complexed with a chelating agent that is conjugated to a linker that is conjugated to B₁₂ or an analog thereof. In still another embodiment, the detectable label may be conjugated to a linker that is conjugated to B₁₂ or an analog thereof. In still yet another embodiment, a detectable label may be indirectly attached to B₁₂ or an analog thereof by the ability of the label to be specifically bound by a second molecule. One example of this type of an indirectly attached label is a biotin label that can be specifically bound by the second molecule, streptavidin. Single, dual or multiple labeling may be advantageous.

As used herein, a “detectable label” is any type of label which, when attached to B₁₂ or an analog thereof renders B₁₂ or the analog thereof detectable. A detectable label may also be toxic to cells or cytotoxic. Accordingly, a detectable label may also be a therapeutic agent or cytotoxic agent. In general, detectable labels may include luminescent molecules, chemiluminescent molecules, fluorochromes, fluorophores, fluorescent quenching agents, colored molecules, radioisotopes, radionuclides, cintillants, massive labels such as a metal atom (for detection via mass changes), biotin, avidin, streptavidin, protein A, protein G, antibodies or fragments thereof, Grb2, polyhistidine, Ni²⁺, Flag tags, myc tags, heavy metals, enzymes, alkaline phosphatase, peroxidase, luciferase, electron donors/acceptors, acridinium esters, and colorimetric substrates. The skilled artisan would readily recognize other useful labels that are not mentioned above, which may be employed in the operation of the present invention.

A detectable label emits a signal that can be detected by a signal transducing machine. In some cases, the detectable label can emit a signal spontaneously, such as when the detectable label is a radionuclide. In other cases the detectable label emits a signal as a result of being stimulated by an external field such as when the detectable label is a relaxivity metal. Examples of signals include, without limitation, gamma rays, X-rays, visible light, infrared energy, and radiowaves. Examples of signal transducing machines include, without limitation, gamma cameras including SPECT/CT devices, PET scanners, fluorimeters, and Magnetic Resonance Imaging (MRI) machines. As such, the detectable label comprises a label that can be detected using magnetic resonance imaging, scintigraphic imaging, ultrasound, or fluorescence. In a specific embodiment, the detectable label comprises a label that can be detected using positron emission tomography, single photon emission computed tomography, gamma camera imaging, or rectilinear scanning.

Suitable fluorophores include, but are not limited to, fluorescein isothiocyante (FITC), fluorescein thiosemicarbazide, rhodamine, Texas Red, CyDyes (e.g., Cy3, Cy5, Cy5.5), Alexa Fluors (e.g., Alexa488, Alexa555, Alexa594; Alexa647), near infrared (NIR) (700-900 nm) fluorescent dyes, and carbocyanine and aminostyryl dyes. B₁₂ or an analog thereof can be labeled for fluorescence detection by labeling the agent with a fluorophore using techniques well known in the art (see, e.g., Lohse et al., Bioconj Chem 8:503-509 (1997)). For example, many known dyes are capable of being coupled to NH₂-terminal groups. Alternatively, a fluorochrome such as fluorescein may be bound to a lysine residue of a peptide linker. In a specific embodiment, an alkyne modified dye, such an Alexa Fluor dye, may be clicked to an azido modified B₁₂ using, for example, Sharpless click chemistry (Kolb et al., Angew Chem Int Ed 2001; 40: 2004-2021, which incorporated by reference in its entirety).

A radionuclide may be a γ-emitting radionuclide, Auger-emitting radionuclide, β-emitting radionuclide, an α-emitting radionuclide, or a positron-emitting radionuclide. A radionuclide may be a detectable label and/or a therapeutic agent. Non-limiting examples of suitable radionuclides may include carbon-11, nitrogen-13, oxygen-15, fluorine-18, fluorodeoxyglucose-18, phosphorous-32, scandium-47, copper-64, 65 and 67, gallium-67 and 68, bromine-75, 77 and 80m, rubidium-82, strontium-89, zirconium-89, yttrium-86 and 90, ruthenium-95, 97, 103 and 105, rhenium-99m, 101, 105, 186 and 188, technetium-99m, rhodium-105, mercury-107, palladium-109, indium-111, silver-111, indium-113m, lanthanide-114m, tin-117m, tellurium-121m, 122m and 125m, iodine-122, 123, 124, 125, 126, 131 and 133, praseodymium-142, promethium-149, samarium-153, gadolinium-159, thulium-165, 167 and 168, dysprosium-165, holmium-166, lutetium-177, rhenium-186 and 188, iridium-192, platinum-193 and 195m, gold-199, thallium-201, titanium-201, astatine-211, bismuth-212 and 213, lead-212, radium-223, actinium-225, and nitride or oxide forms derived there from. In a specific embodiment, a radionuclide is selected from the group consisting of copper-64, zirconium-89, yttrium-86, yttrium-90, technetium-99m, iodine-125, iodine-131, lutetium-177, rhenium-186 and rhenium-188.

A variety of metal atoms may be used as a detectable label. The metal atom may generally be selected from the group of metal atoms comprised of metals with an atomic number of twenty or greater. For instance, the metal atoms may be calcium atoms, scandium atoms, titanium atoms, vanadium atoms, chromium atoms, manganese atoms, iron atoms, cobalt atoms, nickel atoms, copper atoms, zinc atoms, gallium atoms, germanium atoms, arsenic atoms, selenium atoms, bromine atoms, krypton atoms, rubidium atoms, strontium atoms, yttrium atoms, zirconium atoms, niobium atoms, molybdenum atoms, technetium atoms, ruthenium atoms, rhodium atoms, palladium atoms, silver atoms, cadmium atoms, indium atoms, tin atoms, antimony atoms, tellurium atoms, iodine atoms, xenon atoms, cesium atoms, barium atoms, lanthanum atoms, hafnium atoms, tantalum atoms, tungsten atoms, rhenium atoms, osmium atoms, iridium atoms, platinum atoms, gold atoms, mercury atoms, thallium atoms, lead atoms, bismuth atoms, francium atoms, radium atoms, actinium atoms, cerium atoms, praseodymium atoms, neodymium atoms, promethium atoms, samarium atoms, europium atoms, gadolinium atoms, terbium atoms, dysprosium atoms, holmium atoms, erbium atoms, thulium atoms, ytterbium atoms, lutetium atoms, thorium atoms, protactinium atoms, uranium atoms, neptunium atoms, plutonium atoms, americium atoms, curium atoms, berkelium atoms, californium atoms, einsteinium atoms, fermium atoms, mendelevium atoms, nobelium atoms, or lawrencium atoms. In some embodiments, the metal atoms may be selected from the group comprising alkali metals with an atomic number greater than twenty. In other embodiments, the metal atoms may be selected from the group comprising alkaline earth metals with an atomic number greater than twenty. In one embodiment, the metal atoms may be selected from the group of metals comprising the lanthanides. In another embodiment, the metal atoms may be selected from the group of metals comprising the actinides. In still another embodiment, the metal atoms may be selected from the group of metals comprising the transition metals. In yet another embodiment, the metal atoms may be selected from the group of metals comprising the poor metals. In other embodiments, the metal atoms may be selected from the group comprising gold atoms, bismuth atoms, tantalum atoms, and gadolinium atoms. In preferred embodiments, the metal atoms may be selected from the group comprising metals with an atomic number of 53 (i.e. iodine) to 83 (i.e. bismuth). In an alternative embodiment, the metal atoms may be atoms suitable for magnetic resonance imaging. In another alternative embodiment, the metal atoms may be selected from the group consisting of metals that have a K-edge in the x-ray energy band of CT. Preferred metal atoms include, but are not limited to, manganese, iron, gadolinium, gold, and iodine.

The metal atoms may be metal ions in the form of +1, +2, or +3 oxidation states. For instance, non-limiting examples include Ba²⁺, Bi³⁺, Cs⁺, Ca²⁺, Cr²⁺, Cr³⁺, Cr⁶⁺, Co²⁺, Co³⁺, Cu⁺, Cu²⁺, Cu³⁺, Ga³⁺, Gd³⁺, Au⁺, Au³⁺, Fe²⁺, Fe³⁺, F³⁺, Pb²⁺, Mn²⁺, Mn³⁺, Mn⁴⁺, Mn⁷⁺, Hg²⁺, Ni²⁺, Ni³⁺, Ag⁺, Sr²⁺, Sn²⁺, Sn⁴⁺, and Zn²⁺. The metal atoms may comprise a metal oxide. For instance, non-limiting examples of metal oxides may include iron oxide, manganese oxide, or gadolinium oxide. Additional examples may include magnetite, maghemite, or a combination thereof.

In an aspect, B₁₂ or an analog thereof conjugated directly or indirectly to a chelating agent may incorporate a radionuclide or metal atom. Incorporation of the radionuclide or metal atom with B₁₂ or an analog thereof-chelating agent complex may be achieved by various methods common in the art of coordination chemistry. For example, when the metal is technetium-99m, the following general procedure may be used to form a technetium complex. B₁₂ or an analog thereof-chelating agent complex solution is formed initially by dissolving the complex in aqueous alcohol such as ethanol. The solution is then degassed to remove oxygen then thiol protecting groups are removed with a suitable reagent, for example, with sodium hydroxide, and then neutralized with an organic acid, such as acetic acid (pH 6.0-6.5). In the labeling step, a stoichiometric excess of sodium pertechnetate, obtained from a molybdenum generator, is added to a solution of the complex with an amount of a reducing agent such as stannous chloride sufficient to reduce technetium and heated. The labeled complex may be separated from contaminants ^{99m}TcO₄⁻ and colloidal ^{99m}TcO₂ chromatographically, for example, with a C-18 Sep Pak cartridge.

In an alternative method, labeling can be accomplished by a transchelation reaction. The technetium source is a solution of technetium complexed with labile ligands facilitating ligand exchange with the selected chelator. Suitable ligands for transchelation include glycine, tartarate, citrate, and heptagluconate. In this instance the preferred reducing reagent is sodium dithionite. It will be appreciated that the complex may be labeled using the techniques described above, or alternatively the chelator itself may be labeled and subsequently conjugated to B₁₂ or an analog thereof to form the complex; a process referred to as the “prelabeled ligand” method.

Another approach for labeling complexes of the present invention involves immobilizing the B₁₂ or an analog thereof-chelating agent complex on a solid-phase support through a linkage that is cleaved upon metal chelation. This is achieved when the chelating agent is coupled to a functional group of the support by one of the complexing atoms. Preferably, a complexing sulfur atom is coupled to the support which is functionalized with a sulfur protecting group such as maleimide.

In another embodiment, a detectable label may be conjugated directly or indirectly to B₁₂ or an analog thereof without the use of a chelating agent. For example, the detectable label is conjugated directly to B₁₂ or an analog thereof. Or, the detectable label is conjugated to a linker that is conjugated to B₁₂ or an analog thereof. For example, a radioactive iodine label (e.g., ¹²²I, ¹²³I, ¹²⁴I, ¹²⁵I or ¹³¹I) is capable of being conjugated to each D- or L-Tyr or D- or L-4-amino-Phe residue present in a peptide linker. In an embodiment, a tyrosine residue of a peptide linker may be halogenated. Halogens include fluorine, chlorine, bromine, iodine, and astatine. Such halogenated B₁₂s or analogs thereof may be detectably labeled if the halogen is a radioisotope, such as, for example, ¹⁸F, ⁷⁸Br, ⁷⁷Br, ¹²²I, ¹²³I, ¹²⁴I, ¹²⁵I, ¹²⁹I, ¹³¹I or ²¹¹At. Halogenated B₁₂s or analogs thereof contain a halogen covalently bound to at least one amino acid, and preferably to D-Tyr residues present in a peptide linker.

### (c) Therapeutic Agent

In an aspect, B₁₂ or an analog thereof may be conjugated to a therapeutic agent, such that the therapeutic agent can be selectively targeted to a cell expressing cubilin. In a specific embodiment, the therapeutic agent can be selectively targeted to a tumor cell expressing cubilin. The therapeutic agent may be directly conjugated to B₁₂ or an analog thereof or may be indirectly conjugated to B₁₂ or an analog thereof. In an embodiment, the therapeutic agent may be complexed with a chelating agent that is conjugated to B₁₂ or an analog thereof. In another embodiment, the therapeutic agent may be complexed with a chelating agent that is conjugated to a linker that is conjugated to B₁₂ or an analog thereof. In still another embodiment, the therapeutic agent may be conjugated to a linker that is conjugated to B₁₂ or an analog thereof. In still yet another embodiment, the therapeutic agent may be conjugated to a linker that is conjugated to a chelating agent that is complexed with a detectable label and conjugated to B₁₂.

A “therapeutic agent” is any compound known in the art that is used in the detection, diagnosis, or treatment of a condition or disease. Such compounds may be naturally-occurring, modified, or synthetic. Non-limiting examples of therapeutic agents may include drugs, therapeutic compounds, genetic materials, metals (such as radioactive isotopes), proteins, peptides, carbohydrates, lipids, steroids, nucleic acid based materials, or derivatives, analogues, or combinations thereof in their native form or derivatized with hydrophobic or charged moieties to enhance incorporation or adsorption into a cell. Such therapeutic agents may be water soluble or may be hydrophobic. Non-limiting examples of therapeutic agents may include immune-related agents, thyroid agents, respiratory products, antineoplastic agents, anti-helmintics, anti-malarials, mitotic inhibitors, hormones, anti-protozoans, anti-tuberculars, cardiovascular products, blood products, biological response modifiers, anti-fungal agents, vitamins, peptides, anti-allergic agents, anti-coagulation agents, circulatory drugs, metabolic potentiators, anti-virals, anti-anginals, antibiotics, anti-inflammatories, anti-rheumatics, narcotics, cardiac glycosides, neuromuscular blockers, sedatives, local anesthetics, general anesthetics, or radioactive atoms or ions. Non-limiting examples of therapeutic agents are described below. In a specific embodiment, a therapeutic agent may be a compound used in the detection diagnosis or treatment of cancer. The therapeutic agent preferably reduces or interferes with tumor growth or otherwise reduces the effect of the tumor within the body or organism. A therapeutic agent that reduces the symptoms produced by the tumor or reduces tumor growth is suitable for the present invention. Additionally, any therapeutic agent that reduces the symptoms associated with tumor cell growth will work for purposes of the present invention.

B₁₂ or an analog thereof may be conjugated to one, two, three, four, or five therapeutic agents. A linker may or may not be used to conjugate a therapeutic agent to B₁₂ or an analog thereof. Generally speaking, the conjugation should not interfere with intrinsic factor binding to B₁₂ or an analog thereof and also should not interfere with B₁₂-IF binding to cubilin. In some instances, B₁₂ or an analog thereof may be generated with a cleavable linkage between the B₁₂ or analog thereof and therapeutic agent. Such a linker may allow release of the therapeutic agent at a specific cellular location.

A therapeutic agent of the invention may be a small molecule therapeutic, a therapeutic antibody, a therapeutic nucleic acid, or a chemotherapeutic agent. Non-limiting examples of therapeutic antibodies may include muromomab, abciximab, rituximab, daclizumab, basiliximab, palivizumab, infliximab, trastuzumab, etanercept, gemtuzumab, alemtuzumab, ibritomomab, adalimumab, alefacept, omalizumab, tositumomab, efalizumab, cetuximab, bevacizumab, natalizumab, ranibizumab, panitumumab, eculizumab, and certolizumab. A representative therapeutic nucleic acid may encode a polypeptide having an ability to induce an immune response and/or an anti-angiogenic response in vivo. Representative therapeutic proteins with immunostimulatory effects include but are not limited to cytokines (e.g., an interleukin (IL) such as IL2, IL4, IL7, IL12, interferons, granulocyte-macrophage colony-stimulating factor (GM-CSF), tumor necrosis factor alpha (TNF-α)), immunomodulatory cell surface proteins (e.g., human leukocyte antigen (HLA proteins), co-stimulatory molecules, and tumor-associated antigens. See Kirk & Mule, 2000; Mackensen et al., 1997; Walther & Stein, 1999; and references cited therein. Representative proteins with anti-angiogenic activities that can be used in accordance with the presently disclosed subject matter include: thrombospondin I (Kosfeld & Frazier, 1993; Tolsma et al., 1993; Dameron et al., 1994), metallospondin proteins (Carpizo & Iruela-Arispe, 2000), class I interferons (Albini et al., 2000), IL12 (Voest et al., 1995), protamine (Ingber et al., 1990), angiostatin (O'Reilly et al., 1994), laminin (Sakamoto et al., 1991), endostatin (O'Reilly et al., 1997), and a prolactin fragment (Clapp et al., 1993). In addition, several anti-angiogenic peptides have been isolated from these proteins (Maione et al., 1990; Eijan et al., 1991; Woltering et al., 1991). Representative proteins with both immunostimulatory and anti-angiogenic activities may include IL12, interferon-γ, or a chemokine. Other therapeutic nucleic acids that may be useful for cancer therapy include but are not limited to nucleic acid sequences encoding tumor suppressor gene products/antigens, antimetabolites, suicide gene products, and combinations thereof.

A chemotherapeutic agent refers to a chemical compound that is useful in the treatment of cancer. The compound may be a cytotoxic agent that affects rapidly dividing cells in general, or it may be a targeted therapeutic agent that affects the deregulated proteins of cancer cells. A cytotoxic agent is any naturally-occurring, modified, or synthetic compound that is toxic to tumor cells. Such agents are useful in the treatment of neoplasms, and in the treatment of other symptoms or diseases characterized by cell proliferation or a hyperactive cell population. The chemotherapeutic agent may be an alkylating agent, an anti-metabolite, an anti-tumor antibiotic, an anti-cytoskeletal agent, a topoisomerase inhibitor, an anti-hormonal agent, a targeted therapeutic agent, a photodynamic therapeutic agent, or a combination thereof. In an exemplary embodiment, the chemotherapeutic agent is selected from the group consisting of liposomal doxorubicin and nanoparticle albumin docetaxel.

Non-limiting examples of suitable alkylating agents may include altretamine, benzodopa, busulfan, carboplatin, carboquone, carmustine (BCNU), chlorambucil, chlornaphazine, cholophosphamide, chlorozotocin, cisplatin, cyclosphosphamide, dacarbazine (DTIC), estramustine, fotemustine, ifosfamide, improsulfan, lipoplatin, lomustine (CCNU), mafosfamide, mannosulfan, mechlorethamine, mechlorethamine oxide hydrochloride, melphalan, meturedopa, mustine (mechlorethamine), mitobronitol, nimustine, novembichin, oxaliplatin, phenesterine, piposulfan, prednimustine, ranimustine, satraplatin, semustine, temozolomide, thiotepa, treosulfan, triaziquone, triethylenemelamine, triethylenephosphoramide (TEPA), triethylenethiophosphaoramide (thiotepa), trimethylolomelamine, trofosfamide, uracil mustard and uredopa.

Suitable anti-metabolites may include, but are not limited to aminopterin, ancitabine, azacitidine, 8-azaguanine, 6-azauridine, capecitabine, carmofur (1-hexylcarbomoyl-5-fluorouracil), cladribine, clofarabine, cytarabine (cytosine arabinoside (Ara-C)), decitabine, denopterin, dideoxyuridine, doxifluridine, enocitabine, floxuridine, fludarabine, 5-fluorouracil, gemcitabine, hydroxyurea (hydroxycarbamide), leucovorin (folinic acid), 6-mercaptopurine, methotrexate, nafoxidine, nelarabine, oblimersen, pemetrexed, pteropterin, raltitrexed, tegofur, tiazofurin, thiamiprine, tioguanine (thioguanine), and trimetrexate.

Non-limiting examples of suitable anti-tumor antibiotics may include aclacinomysin, aclarubicin, actinomycins, adriamycin, aurostatin (for example, monomethyl auristatin E), authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, epoxomicin, esorubicin, idarubicin, marcellomycin, mitomycins, mithramycin, mycophenolic acid, nogalamycin, olivomycins, peplomycin, plicamycin, potfiromycin, puromycin, quelamycin, rodorubicin, sparsomycin, streptonigrin, streptozocin, tubercidin, valrubicin, ubenimex, zinostatin, and zorubicin.

Non-limiting examples of suitable anti-cytoskeletal agents may include cabazitaxel, colchicines, demecolcine, docetaxel, epothilones, ixabepilone, macromycin, omacetaxine mepesuccinate, ortataxel, paclitaxel (for example, DHA-paclitaxel), taxane, tesetaxel, vinblastine, vincristine, vindesine, and vinorelbine.

Suitable topoisomerase inhibitors may include, but are not limited to, amsacrine, etoposide (VP-16), irinotecan, mitoxantrone, RFS 2000, teniposide, and topotecan.

Non-limiting examples of suitable anti-hormonal agents may include aminoglutethimide, antiestrogen, aromatase inhibiting 4(5)-imidazoles, bicalutamide, finasteride, flutamide, fluvestrant, goserelin, 4-hydroxytamoxifen, keoxifene, leuprolide, LY117018, mitotane, nilutamide, onapristone, raloxifene, tamoxifen, toremifene, and trilostane.

Examples of targeted therapeutic agents may include, without limit, monoclonal antibodies such as alemtuzumab, cartumaxomab, edrecolomab, epratuzumab, gemtuzumab, gemtuzumab ozogamicin, glembatumumab vedotin, ibritumomab tiuxetan, reditux, rituximab, tositumomab, and trastuzumab; protein kinase inhibitors such as bevacizumab, cetuximab, crizonib, dasatinib, erlotinib, gefitinib, imatinib, lapatinib, mubritinib, nilotinib, panitumumab, pazopanib, sorafenib, sunitinib, toceranib, and vandetanib.

Non limiting examples of angiogeneisis inhibitors may include angiostatin, bevacizumab, denileukin diftitox, endostatin, everolimus, genistein, interferon alpha, interleukin-2, interleukin-12, pazopanib, pegaptanib, ranibizumab, rapamycin (sirolimus), temsirolimus, and thalidomide.

Non limiting examples of growth inhibitory polypeptides may include bortazomib, erythropoietin, interleukins (e.g., IL-1, IL-2, IL-3, IL-6), leukemia inhibitory factor, interferons, romidepsin, thrombopoietin, TNF-α, CD30 ligand, 4-1 BB ligand, and Apo-1 ligand.

Non-limiting examples of photodynamic therapeutic agents may include aminolevulinic acid, methyl aminolevulinate, retinoids (alitretinon, tamibarotene, tretinoin), and temoporfin.

Other antineoplastic agents may include anagrelide, arsenic trioxide, asparaginase, bexarotene, bropirimine, celecoxib, chemically linked Fab, efaproxiral, etoglucid, ferruginol, lonidamide, masoprocol, miltefosine, mitoguazone, talapanel, trabectedin, and vorinostat.

Also included are pharmaceutically acceptable salts, acids, or derivatives of any of the above listed agents. The dose of the chemotherapeutic agent can and will vary depending upon the agent and the type of tumor or neoplasm. A skilled practitioner will be able to determine the appropriate dose of the chemotherapeutic agent.

Other therapeutic agents may comprise a virus or a viral genome such as an oncolytic virus. An oncolytic virus comprises a naturally occurring virus that is capable of killing a cell in the target tissue (for example, by lysis) when it enters such a cell.

### (d) Intrinsic Factor

Intrinsic factor (IF) is a glycosylated protein that is secreted from the gastric mucosa and the pancreas. IF binds B₁₂ with picomolar affinity (K_{d}˜1 pM). In the B₁₂ uptake pathway, the IF protein facilitates transport of B₁₂ across the intestinal enterocyte, which occurs by receptor-mediated endocytosis at the apically expressed IF-B₁₂ receptor (cubilin). Cubilin works to transport B₁₂ in concert with an anchoring protein amnionless (Am). Following transcytosis, and between 2.5 and 4 h after initial ingestion, B₁₂ appears in blood plasma bound to the third trafficking protein, transcobalamin II (TCII). Based on the described pathway of B₁₂ uptake, IF is not present in the blood and instead present only in the gastrointestinal tract. Accordingly, prior to the disclosure, it was unknown that IF-B₁₂ could be administered via a non-oral route (i.e. the blood stream) and effectively bind cubilin. As such, the inventors unexpectedly discovered that IF-B₁₂ administered intravenously can bind to cubilin outside of the gastrointestinal tract (i.e. non-ileal cubilin).

In an aspect, IF is bound to B₁₂ or to a B₁₂ conjugate of the invention thereby forming a complex. The IF may be bound to B₁₂ or analog thereof before or after conjugation of B₁₂ or an analog thereof to a detectable label and/or therapeutic. In a specific embodiment, IF may be bound to B₁₂ or an analog thereof after conjugation of B₁₂ or an analog thereof to a detectable label and/or therapeutic. In an embodiment, IF may be pre-bound to a B₁₂ or B₁₂ conjugate by combining the conjugate with IF in solution. By way of non-limiting example, B₁₂ or B₁₂ conjugate may be combined with IF in PBS at pH 7.4 or in MES buffer at pH 5.5 or in water at pH 8 at temperatures ranging from about 25° C. to about 37° C. For binding, IF may be contacted with B₁₂ or B₁₂ conjugate for at least 30 minutes. Alternatively, IF may be contacted with B₁₂ or B₁₂ conjugate for at least 15 minutes, at least 30 minutes, at least 45 minutes, at least 1 hour, at least 2 hours, at least 3 hours, at least 4 hours, at least 5 hours or at least 6 hours. A skilled artisan would be able to determine the various conditions upon which IF and B₁₂ or B₁₂ conjugate may be pre-bound.

For pre-binding of IF and B₁₂ or the B₁₂ conjugate, IF and B₁₂ or the B₁₂ conjugate may be combined in solution. One IF binds to one B₁₂ or B₁₂ conjugate. Accordingly, the ratio of IF to B₁₂ or B₁₂ conjugate added to solution may be 1:1. However, to facilitate saturation of the IF with B₁₂ or B₁₂ conjugate, a greater amount of IF may be added to solution relative to B₁₂ or B₁₂ conjugate. For example, the ratio of IF to B₁₂ or B₁₂ conjugate may be 1.1:1, 1.2:1, 1.3:1, 1.4:1, 1.5:1, 2:1, 2.5:1, 3:1, 3.5:1, 4:1, 4.5:1, 5:1, 6:1, 7:1, 8:1, 9:1, 10:1, 15:1, 20:1, 25:1, 30:1, 35:1, 40:1, 45:1, 50:1, 60:1, 70:1, 80:1, 90:1, or 100:1. In specific embodiments, the ratio of IF to B₁₂ or B₁₂ conjugate may be 1.1:1, 1.2:1, 1.3:1, 1.4:1, 1.5:1, 2:1, 2.5:1, 3:1, 3.5:1, 4:1, 4.5:1, 5:1, 6:1, 7:1, 8:1, 9:1, or 10:1. In other embodiments, an excess of 5% or more IF relative to B₁₂ or B₁₂ conjugate may be added to solution. For example, an excess of 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 70%, 80%, 90% or 100% IF relative to B₁₂ or B₁₂ conjugate may be added to solution. In specific embodiments, an excess of 5%, 6%, 7%, 8%, 9%, 10%, 11%, 12%, 13%, 14%, or 15% IF relative to B₁₂ or B₁₂ conjugate may be added to solution. Preferably, in some embodiments, excess IF is added to the solution relative to B₁₂ or B₁₂ conjugate. However, it may be necessary to add a greater amount of B₁₂ or B₁₂ conjugate relative to IF to reduce or eliminate unbound IF. Accordingly, the ratio of B₁₂ or B₁₂ conjugate to IF may be 1.1:1, 1.2:1, 1.3:1, 1.4:1, 1.5:1, 2:1, 2.5:1, 3:1, 3.5:1, 4:1, 4.5:1, 5:1, 6:1, 7:1, 8:1, 9:1, 10:1, 15:1, 20:1, 25:1, 30:1, 35:1, 40:1, 45:1, 50:1, 60:1, 70:1, 80:1, 90:1, or 100:1. In specific embodiments, the ratio of B₁₂ or B₁₂ conjugate to IF may be 1.1:1, 1.2:1, 1.3:1, 1.4:1, 1.5:1, 2:1, 2.5:1, 3:1, 3.5:1, 4:1, 4.5:1, 5:1, 6:1, 7:1, 8:1, 9:1, or 10:1. In other embodiments, an excess of 5% or more B₁₂ or B₁₂ conjugate relative to IF may be added to solution. For example, an excess of 5%, 6%, 7%, 8%, 9%, 10%, 11%, 12%, 13%, 14%, 15%, 16%, 17%, 18%, 19%, 20%, 25%, 30%, 35%, 40%, 45%, 50%, 55%, 60%, 70%, 80%, 90% or 100% B₁₂ or B₁₂ conjugate relative to IF may be added to a solution. In a specific embodiment, an excess of 5%, 6%, 7%, 8%, 9%, 10%, 11%, 12%, 13%, 14%, or 15% B₁₂ or B₁₂ conjugate relative to IF may be added to a solution. Prior to administration of a composition of the invention, it may be necessary to remove unbound IF and/or unbound B₁₂ or B₁₂ conjugate. In the case of imaging, removal of unbound B₁₂ or B₁₂ conjugate may be necessary to reduce background.

IF of the invention may be expressed and purified via standard methodology. In a specific embodiment, IF may be expressed and purified from a transgenic plant, such as Arabidopsis. The expressed and purified IF may be from any species, provided it binds to B₁₂ or a B₁₂ conjugate and human cubilin. A skilled artisan will appreciate that IF can be found in a variety of species. Non-limiting examples include human (NP_005133.2), mouse (P52787.2), rat (NP_058858.1), dog (Q5XWD5.1), cat (XP_003993466.1), cattle (NP_001193168.1), non-human primates (EHH56203.1, XP_004051305.1), and horse (XP_008508117.1). It is appreciated that the present invention is directed to homologs of IF in other organisms and is not limited to the human protein. Homologs can be found in other species by methods known in the art. For example, sequence similarity may be determined by conventional algorithms, which typically allow introduction of a small number of gaps in order to achieve the best fit. In particular, “percent identity” of two polypeptides or two nucleic acid sequences is determined using the algorithm of Karlin and Altschul (Proc. Natl. Acad. Sci. USA 87:2264-2268, 1993). Such an algorithm is incorporated into the BLASTN and BLASTX programs of Altschul et al. (J. Mol. Biol. 215:403-410, 1990). BLAST nucleotide searches may be performed with the BLASTN program to obtain nucleotide sequences homologous to a nucleic acid molecule of the invention. Equally, BLAST protein searches may be performed with the BLASTX program to obtain amino acid sequences that are homologous to a polypeptide of the invention. To obtain gapped alignments for comparison purposes, Gapped BLAST is utilized as described in Altschul et al. (Nucleic Acids Res. 25:3389-3402, 1997). When utilizing BLAST and Gapped BLAST programs, the default parameters of the respective programs (e.g., BLASTX and BLASTN) are employed. See www.ncbi.nlm.nih.gov for more details. In some embodiments, a homolog has at least 80%, at least 81%, at least 82%, at least 83%, at least 84%, at least 85%, at least 86%, at least 87%, at least 88%, or 89% identity to human IF. In another embodiment %, a homolog has at least 90%, at least 91 at least %, at least 92 at least %, at least 93 at least %, at least 94%, at least 95%, at least 96%, at least 97%, at least 98%, at least 99%, or at least 100% identity to human IF. For instance, a homolog may have at least 80%, at least 81%, at least 82%, at least 83%, at least 84%, at least 85%, at least 86%, at least 87%, at least 88%, or 89% identity to human IF. In another embodiment %, a homolog has at least 90%, at least 91 at least %, at least 92 at least %, at least 93 at least %, at least 94%, at least 95%, at least 96%, at least 97%, at least 98%, at least 99%, or at least 100% identity to the IF sequence accession number NP_005133.2.

In a specific embodiment, the IF comprises the sequence disclosed in accession number NP_005133.2. In other embodiments, the IF comprises the sequence disclosed in accession number NP 005133.2 but for one to 10 conservative amino acid substitutions. For example, the IF comprises the sequence disclosed in accession number NP_005133.2 but for 1, 2, 3, 4, 5, 6, 7, 8, 9 or 10 conservative amino acid substitutions. As used herein, a “conservative amino acid substitution” is one in which the amino acid residue is replaces with an amino acid residue having a similar side chain. Families of amino acid residues having similar side chains have been defined in the art. These families include amino acids with basic side chains (e.g. lysine, arginine, histidine), acidic side chains (e.g., aspartic acid, glutamic acid), uncharged polar side chains (e.g., asparagine, glutamine, serine, threonine, tyrosine, cysteine), nonpolar side chains (e.g. glycine, alanine, valine, leucine, isoleucine, proline, phenylalanine, methionine, tryptophan, histidine). The resulting peptide comprising the substitution should have similar characteristics or properties including size, hydrophobicity, etc., such that the overall functionally of the peptide does not significantly change. As the structure of IF bound to B₁₂ is known in the art, a skilled artisan would be able to determine amino acids essential to B₁₂ binding to ensure binding to B₁₂ or a B₁₂ conjugate.

### (e) Pharmaceutical Formulation

The present disclosure also provides pharmaceutical formulations for parenteral administration. The pharmaceutical formulation comprises intrinisic factor and B₁₂ or a B₁₂ conjugate, as an active ingredient, and at least one pharmaceutically acceptable carrier for parenteral administration. The term parenteral as used herein includes subcutaneous, intravenous, intramuscular, intradermal, intra-arterial, intraosseous, intraperitoneal, or intrathecal injection, or infusion techniques. In one embodiment, the invention encompasses a formulation for IV administration, the formulation comprising intrinisic factor and B₁₂ or a B₁₂ conjugate, as an active ingredient, and at least one pharmaceutically acceptable carrier for IV administration.

The composition can be formulated into various dosage forms and administered by a number of different means that will deliver a therapeutically effective amount of the active ingredient. Such compositions can be administered parenterally in dosage unit formulations containing conventional nontoxic pharmaceutically acceptable carriers, adjuvants, and vehicles as desired. Formulation of drugs is discussed in, for example, Gennaro, A. R., Remington's Pharmaceutical Sciences, Mack Publishing Co., Easton, Pa. (18^{th} ed, 1995), and Liberman, H. A. and Lachman, L., Eds., Pharmaceutical Dosage Forms, Marcel Dekker Inc., New York, N.Y. (1980).

For parenteral administration, the preparation may be an aqueous or an oil-based solution. Aqueous solutions may include a sterile diluent or excipient such as water, saline solution, a pharmaceutically acceptable polyol such as glycerol, propylene glycol, or other synthetic solvents; an antibacterial and/or antifungal agent such as benzyl alcohol, methyl paraben, chlorobutanol, phenol, thimerosal, and the like; an antioxidant such as ascorbic acid or sodium bisulfite; a chelating agent such as etheylenediaminetetraacetic acid; a buffer such as acetate, citrate, or phosphate; and/or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil. The compositions may be presented in unit-dose or multi-dose containers, for example sealed ampoules and vials, and may be stored in a freeze-dried (lyophilized) condition requiring only the addition of the sterile liquid carried, for example water for injections, immediately prior to use. Extemporaneous injection solutions and suspensions may be prepared from sterile powders, granules and tablets.

In certain embodiments, a composition comprising IF and a B₁₂ conjugate is encapsulated in a suitable vehicle to either aid in the delivery of the compound to target cells, to increase the stability of the composition, or to minimize potential toxicity of the composition. As will be appreciated by a skilled artisan, a variety of vehicles are suitable for delivering a composition of the present invention. Non-limiting examples of suitable structured fluid delivery systems may include nanoparticles, liposomes, microemulsions, micelles, dendrimers and other phospholipid-containing systems. Methods of incorporating compositions into delivery vehicles are known in the art.

In one alternative embodiment, a liposome delivery vehicle may be utilized. Liposomes, depending upon the embodiment, are suitable for delivery of the IF and B₁₂ conjugate in view of their structural and chemical properties. Generally speaking, liposomes are spherical vesicles with a phospholipid bilayer membrane. The lipid bilayer of a liposome may fuse with other bilayers (e.g., the cell membrane), thus delivering the contents of the liposome to cells. In this manner, the IF and B₁₂ conjugate may be selectively delivered to a cell by encapsulation in a liposome that fuses with the targeted cell's membrane.

Liposomes may be comprised of a variety of different types of phosolipids having varying hydrocarbon chain lengths. Phospholipids generally comprise two fatty acids linked through glycerol phosphate to one of a variety of polar groups. Suitable phospholids include phosphatidic acid (PA), phosphatidylserine (PS), phosphatidylinositol (PI), phosphatidylglycerol (PG), diphosphatidylglycerol (DPG), phosphatidylcholine (PC), and phosphatidylethanolamine (PE). The fatty acid chains comprising the phospholipids may range from about 6 to about 26 carbon atoms in length, and the lipid chains may be saturated or unsaturated. Suitable fatty acid chains include (common name presented in parentheses) n-dodecanoate (laurate), n-tretradecanoate (myristate), n-hexadecanoate (palmitate), n-octadecanoate (stearate), n-eicosanoate (arachidate), n-docosanoate (behenate), n-tetracosanoate (lignocerate), cis-9-hexadecenoate (palmitoleate), cis-9-octadecanoate (oleate), cis,cis-9,12-octadecandienoate (linoleate), all cis-9, 12, 15-octadecatrienoate (linolenate), and all cis-5,8,11,14-eicosatetraenoate (arachidonate). The two fatty acid chains of a phospholipid may be identical or different. Acceptable phospholipids include dioleoyl PS, dioleoyl PC, distearoyl PS, distearoyl PC, dimyristoyl PS, dimyristoyl PC, dipalmitoyl PG, stearoyl, oleoyl PS, palmitoyl, linolenyl PS, and the like.

The phospholipids may come from any natural source, and, as such, may comprise a mixture of phospholipids. For example, egg yolk is rich in PC, PG, and PE, soy beans contains PC, PE, PI, and PA, and animal brain or spinal cord is enriched in PS. Phospholipids may come from synthetic sources too. Mixtures of phospholipids having a varied ratio of individual phospholipids may be used. Mixtures of different phospholipids may result in liposome compositions having advantageous activity or stability of activity properties. The above mentioned phospholipids may be mixed, in optimal ratios with cationic lipids, such as N-(1-(2,3-dioleolyoxy)propyl)-N,N,N-trimethyl ammonium chloride, 1,1′-dioctadecyl-3,3,3′,3′-tetramethylindocarbocyanine perchloarate, 3,3′-deheptyloxacarbocyanine iodide, 1,1′-dedodecyl-3,3,3′,3′-tetramethylindocarbocyanine perchloarate, 1,1′-dioleyl-3,3,3′,3′-tetramethylindo carbocyanine methanesulfonate, N-4-(delinoleylaminostyryl)-N-methylpyridinium iodide, or 1,1,-dilinoleyl-3,3,3′,3′-tetramethylindocarbocyanine perchloarate.

Liposomes may optionally comprise sphingolipids, in which spingosine is the structural counterpart of glycerol and one of the one fatty acids of a phosphoglyceride, or cholesterol, a major component of animal cell membranes. Liposomes may optionally contain pegylated lipids, which are lipids covalently linked to polymers of polyethylene glycol (PEG). PEGs may range in size from about 500 to about 10,000 daltons.

Liposomes may further comprise a suitable solvent. The solvent may be an organic solvent or an inorganic solvent. Suitable solvents include, but are not limited to, dimethylsulfoxide (DMSO), methylpyrrolidone, N-methylpyrrolidone, acetronitrile, alcohols, dimethylformamide, tetrahydrofuran, or combinations thereof.

Liposomes carrying IF and a B₁₂ conjugate (i.e., having at least one methionine compound) may be prepared by any known method of preparing liposomes for drug delivery, such as, for example, detailed in U.S. Pat. Nos. 4,241,046, 4,394,448, 4,529,561, 4,755,388, 4,828,837, 4,925,661, 4,954,345, 4,957,735, 5,043,164, 5,064,655, 5,077,211 and 5,264,618, the disclosures of which are hereby incorporated by reference in their entirety. For example, liposomes may be prepared by sonicating lipids in an aqueous solution, solvent injection, lipid hydration, reverse evaporation, or freeze drying by repeated freezing and thawing. In a preferred embodiment the liposomes are formed by sonication. The liposomes may be multilamellar, which have many layers like an onion, or unilamellar. The liposomes may be large or small. Continued high-shear sonication tends to form smaller unilamellar lipsomes.

As would be apparent to one of ordinary skill, all of the parameters that govern liposome formation may be varied. These parameters include, but are not limited to, temperature, pH, concentration of methionine compound, concentration and composition of lipid, concentration of multivalent cations, rate of mixing, presence of and concentration of solvent.

In another embodiment, a composition of the invention may be delivered to a cell as a microemulsion. Microemulsions are generally clear, thermodynamically stable solutions comprising an aqueous solution, a surfactant, and “oil.” The “oil” in this case, is the supercritical fluid phase. The surfactant rests at the oil-water interface. Any of a variety of surfactants are suitable for use in microemulsion formulations including those described herein or otherwise known in the art. The aqueous microdomains suitable for use in the invention generally will have characteristic structural dimensions from about 5 nm to about 100 nm. Aggregates of this size are poor scatterers of visible light and hence, these solutions are optically clear. As will be appreciated by a skilled artisan, microemulsions can and will have a multitude of different microscopic structures including sphere, rod, or disc shaped aggregates. In one embodiment, the structure may be micelles, which are the simplest microemulsion structures that are generally spherical or cylindrical objects. Micelles are like drops of oil in water, and reverse micelles are like drops of water in oil. In an alternative embodiment, the microemulsion structure is the lamellae. It comprises consecutive layers of water and oil separated by layers of surfactant. The “oil” of microemulsions optimally comprises phospholipids. Any of the phospholipids detailed above for liposomes are suitable for embodiments directed to microemulsions. The IF and B₁₂ conjugate may be encapsulated in a microemulsion by any method generally known in the art.

In yet another embodiment, IF and a B₁₂ conjugate may be delivered in a dendritic macromolecule, or a dendrimer. Generally speaking, a dendrimer is a branched tree-like molecule, in which each branch is an interlinked chain of molecules that divides into two new branches (molecules) after a certain length. This branching continues until the branches (molecules) become so densely packed that the canopy forms a globe. Generally, the properties of dendrimers are determined by the functional groups at their surface. For example, hydrophilic end groups, such as carboxyl groups, would typically make a water-soluble dendrimer. Alternatively, phospholipids may be incorporated in the surface of a dendrimer to facilitate absorption across the skin. Any of the phospholipids detailed for use in liposome embodiments are suitable for use in dendrimer embodiments. Any method generally known in the art may be utilized to make dendrimers and to encapsulate compositions of the invention therein. For example, dendrimers may be produced by an iterative sequence of reaction steps, in which each additional iteration leads to a higher order dendrimer. Consequently, they have a regular, highly branched 3D structure, with nearly uniform size and shape. Furthermore, the final size of a dendrimer is typically controlled by the number of iterative steps used during synthesis. A variety of dendrimer sizes are suitable for use in the invention. Generally, the size of dendrimers may range from about 1 nm to about 100 nm.

### II. Methods

In another aspect, a composition of the present invention, as described above, may be used in treating, stabilizing and preventing cancer and associated diseases in a subject. By “treating, stabilizing, or preventing cancer” is meant causing a reduction in the size of a tumor or in the number of cancer cells, slowing or preventing an increase in the size of a tumor or cancer cell proliferation, increasing the disease-free survival time between the disappearance of a tumor or other cancer and its reappearance, preventing an initial or subsequent occurrence of a tumor or other cancer, or reducing an adverse symptom associated with a tumor or other cancer. In a desired embodiment, the percent of tumor or cancerous cells surviving the treatment is at least 20, 40, 60, 80, or 100% lower than the initial number of tumor or cancerous cells, as measured using any standard assay (e.g., caspase assays, TUNEL and DNA fragmentation assays, cell permeability assays, and Annexin V assays). Desirably, the decrease in the number of tumor or cancerous cells induced by administration of a composition of the invention is at least 2, 5, 10, 20, or 50-fold greater than the decrease in the number of non-tumor or non-cancerous cells. Desirably, the methods of the present invention result in a decrease of 20, 40, 60, 80, or 100% in the size of a tumor or in the number of cancerous cells, as determined using standard methods. Desirably, at least 20, 40, 60, 80, 90, or 95% of the treated subjects have a complete remission in which all evidence of the tumor or cancer disappears. Desirably, the tumor or cancer does not reappear or reappears after at least 5, 10, 15, or 20 years.

The B₁₂ of the present invention may be indirectly or directly conjugated to radionuclides or therapeutic agents as described above in order to provide specific delivery of radiation and therapy to the site of a tumor. For example, the IF-B₁₂ conjugate administered intravenously binds cubilin outside the gastrointestinal tract. The IF-B₁₂ conjugate is then internalized and the detectable label and/or therapeutic agent is accumulated in cells expressing cubilin. By this mechanism, a composition of the invention may be used to provide specific delivery of radiation and therapy to the site of a tumor. Further, the composition of the present invention may be part of a combination therapy. Preferably, a combination therapy would include the use of the composition of the present invention along with a radiation therapy or chemotherapy course of treatment.

In yet another aspect, the present invention provides a method of detecting a tumor in a subject. The method comprises administering to the subject a composition comprising IF and B₁₂, wherein the B₁₂ is conjugated to a detectable label, and detecting the detectable label to detect binding of the composition to cubilin in the subject, wherein the presence of the detectable label in a tissue that does not typically express cubilin indicates the presence of a tumor in the subject. In another embodiment, the method comprises administering to the subject a composition comprising IF and B₁₂, wherein the B₁₂ is conjugated to a detectable label, and detecting the detectable label to detect binding of the composition to cubilin in the subject, wherein the asymmetrical presence of the detectable label in a tissue that is known to express cubilin indicates the presence of a tumor in the subject. In preferred embodiments, the methods may be used to diagnose or image a cancer or tumor in a subject. In other embodiments, the methods may be used to image cubilin expression outside the gastrointestinal tract in a subject. In some embodiments, a method for detecting a tumor can comprise (a) biopsying a suspected tumor; (c) contacting a composition of the invention with the suspected tumor in vitro; and (d) detecting the detectable label in a tissue that does not typically express cubilin, whereby a tumor is diagnosed.

Binding may be detected using microscopy (fluorescent microscopy, confocal microscopy, or electron microscopy), magnetic resonance imaging (including MTI, MRS, DWI and fMRI), scintigraphic imaging (SPECT (Single Photon Emission Computed Tomography), PET (Positron Emission Tomography), gamma camera imaging, and rectilinear scanning), radiography, or ultrasound. The detectable label may be detectable in situ, in vivo, ex vivo, and in vitro.

In still yet another aspect, the present invention provides a method of delivering B₁₂ to a cell that expresses cubilin in a subject. The method comprises administering a complex of IF and B₁₂ to the subject intravenously. Accordingly, the complex of IF and B₁₂ may bind to cubilin present on a cell thereby delivering B₁₂ to the cell. In an embodiment, the B₁₂ is conjugated to a detectable label and/or therapeutic agent. Such a method may be used to detect or treat a cell that expresses cubilin in a subject.

In yet still another aspect, the present invention provides a method of modulating cubilin function. The method comprises administering a complex of IF and B₁₂ to the subject intravenously. Accordingly, the complex of IF and B₁₂ may bind to cubilin present on a cell thereby modulating cubilin function. By modulate is meant to change the activity of cubilin. For example, the complex may block cubilin function thereby inhibiting the activity of cubilin. Using intravenous administration, the method may modulate the renal proximal tubule reabsorption of filtered proteins including albumin, transferrin, vitamin D-binding protein and other important plasma carriers. For example, administration of a complex of IF and B₁₂ may modulate cubilin function thereby reducing or preventing uptake of nephrotoxic proteins. Reduction or prevention of uptake of nephrotoxic proteins may treat or prevent acute kidney injury. As used herein, the term “kidney injury” refers to a loss of kidney function. The causes of kidney injury known in the art are numerous, and may include, but are not limited to, necrosis, ischemia, vascular damage, exposure to substances that damage the kidney such as toxins, intravenous contrast, antibiotics, pigments, and LPS, obstruction of the urinary tract, and trauma or crush injury to the kidney. Further by “kidney injury” is meant acute kidney injury, as defined according to the Acute Kidney Injury Network criteria (see Metha et al. Cri Care 2007). For example, a complex of IF and B₁₂ may be administered prior to, concurrent with, or after administration of a nephrotoxic drug such that nephrotoxicity is reduced or eliminated. In an embodiment, the B₁₂ is conjugated to a detectable label and/or therapeutic agent.

The composition, B₁₂ and IF are as described in Section I above. Cubulin, the subject, the cancer, and the administration of the composition are described below.

### (a) Cubilin

Cubilin is a large endocytic receptor serving such diverse functions as the intestinal absorption of the IF-B₁₂ complex and the renal proximal tubule reabsorption of filtered proteins including albumin, transferrin, vitamin D-binding protein and other important plasma carriers.

In an aspect, asymmetrical presence of a detectable label in a tissue comprising cells that are known to express cubilin may indicate the presence of a tumor in a subject. In another aspect, the presence of a detectable label in a tissue that comprises cells that do not typically express cubilin indicates the presence of a tumor in a subject. Non-limiting examples of cells that are known to express cubilin include mammary epithelial cells, renal proximal tubular cells, gall bladder cells, gastrointestinal tract cells, brush border cells, placental cells, podocytes, and epithelial cells in the inner ear and of the yolk sac. Methods to detect if a tissue or cell is known to express cubilin or if a tissue or cell does not typically express cubilin are known in the art. Based on these methods, it will be within the ability of a skilled artisan to determine whether the tissue will be examined for asymmetrical presence of a detectable label or the presence of a detectable label. For example, methods to detect protein expression are well known in the art and all suitable methods for assessing an amount of protein expression known to one of skill in the art are contemplated within the scope of the invention. Generally, the method comprises obtaining a tissue sample and processing the sample in vitro to assess the amount of protein expression. Non-limiting examples of suitable methods to assess an amount of protein expression may include epitope binding agent-based methods and mass spectrometry based methods. In some embodiments, the method to assess an amount of protein expression is an epitope binding agent-based method. As used herein, the term “epitope binding agent” refers to an antibody, an aptamer, a nucleic acid, an oligonucleic acid, an amino acid, a peptide, a polypeptide, a protein, a lipid, a metabolite, a small molecule, or a fragment thereof that recognizes and is capable of binding to cubilin. Nucleic acids may include RNA, DNA, and naturally occurring or synthetically created derivative.

As used herein, the term “antibody” generally means a polypeptide or protein that recognizes and can bind to an epitope of cubilin. An antibody, as used herein, may be a complete antibody as understood in the art, i.e., consisting of two heavy chains and two light chains, or may be any antibody-like molecule that has an antigen binding region, and includes, but is not limited to, antibody fragments such as Fab′, Fab, F(ab′)2, single domain antibodies, Fv, and single chain Fv. The term antibody also refers to a polyclonal antibody, a monoclonal antibody, a chimeric antibody and a humanized antibody. The techniques for preparing and using various antibody-based constructs and fragments are well known in the art. Means for preparing and characterizing antibodies are also well known in the art (See, e.g. Antibodies: A Laboratory Manual, Cold Spring Harbor Laboratory, 1988; herein incorporated by reference in its entirety).

As used herein, the term “aptamer” refers to a polynucleotide, generally a RNA or DNA that has a useful biological activity in terms of biochemical activity, molecular recognition or binding attributes. Usually, an aptamer has a molecular activity such as binging to a target molecule at a specific epitope (region). It is generally accepted that an aptamer, which is specific in it binding to a polypeptide, may be synthesized and/or identified by in vitro evolution methods. Means for preparing and characterizing aptamers, including by in vitro evolution methods, are well known in the art (See, e.g. U.S. Pat. No. 7,939,313; herein incorporated by reference in its entirety).

In general, an epitope binding agent-based method of assessing an amount of protein expression comprises contacting a sample with an epitope binding agent specific for cubilin under conditions effective to allow for formation of a complex between the epitope binding agent and cubilin. Epitope binding agent-based methods may occur in solution, or the epitope binding agent or sample may be immobilized on a solid surface. Non-limiting examples of suitable surfaces include microtitre plates, test tubes, beads, resins, and other polymers.

An epitope binding agent may be attached to the substrate in a wide variety of ways, as will be appreciated by those in the art. The epitope binding agent may either be synthesized first, with subsequent attachment to the substrate, or may be directly synthesized on the substrate. The substrate and the epitope binding agent may be derivatized with chemical functional groups for subsequent attachment of the two. For example, the substrate may be derivatized with a chemical functional group including, but not limited to, amino groups, carboxyl groups, oxo groups or thiol groups. Using these functional groups, the epitope binding agent may be attached directly using the functional groups or indirectly using linkers.

The epitope binding agent may also be attached to the substrate non-covalently. For example, a biotinylated epitope binding agent may be prepared, which may bind to surfaces covalently coated with streptavidin, resulting in attachment. Alternatively, an epitope binding agent may be synthesized on the surface using techniques such as photopolymerization and photolithography. Additional methods of attaching epitope binding agents to solid surfaces and methods of synthesizing biomolecules on substrates are well known in the art, i.e. VLSIPS technology from Affymetrix (e.g., see U.S. Pat. No. 6,566,495, and Rockett and Dix, Xenobiotica 30(2):155-177, both of which are hereby incorporated by reference in their entirety).

Contacting the sample with an epitope binding agent under effective conditions for a period of time sufficient to allow formation of a complex generally involves adding the epitope binding agent composition to the sample and incubating the mixture for a period of time long enough for the epitope binding agent to bind to any cubilin present. After this time, the complex will be washed and the complex may be detected by any method well known in the art. Methods of detecting the epitope binding agent-cubilin complex are generally based on the detection of a label or marker. The term “label”, as used herein, refers to any substance attached to an epitope binding agent, or other substrate material, in which the substance is detectable by a detection method. Non-limiting examples of suitable labels include luminescent molecules, chemiluminescent molecules, fluorochromes, fluorescent quenching agents, colored molecules, radioisotopes, scintillants, biotin, avidin, stretpavidin, protein A, protein G, antibodies or fragments thereof, polyhistidine, Ni2+, Flag tags, myc tags, heavy metals, and enzymes (including alkaline phosphatase, peroxidase, and luciferase). Methods of detecting an epitope binding agent-cubilin complex based on the detection of a label or marker are well known in the art.

In some embodiments, the epitope binding agent-based method is an ELISA. In other embodiments, the epitope binding agent-based method is a radioimmunoassay. In still other embodiments, the epitope binding agent-based method is an immunoblot or Western blot. In different embodiments, the epitope binding agent-based method is immunohistochemistry (IHC). In alternative embodiments, the epitope binding agent-based method is an array. In other embodiments, the epitope binding agent-based method is flow cytometry.

By asymmetrical is meant that the detectable label is not dispersed evenly throughout the tissue. Instead, asymmetrical may mean that the detectable label is accumulated in a portion of the tissue or randomly localized to a portion of the tissue. In an embodiment, asymmetrical presence of the detectable label may mean a difference in signal of about 5% or more relative to the rest of the tissue. For example, asymmetrical presence of the detectable label may mean a difference in signal of greater than about 5%, greater than about 10%, greater than about 15%, greater than about 20%, greater than about 25%, greater than about 30%, greater than about 35%, greater than about 40%, greater than about 45%, greater than about 50%, greater than about 55%, greater than about 60%, greater than about 65%, greater than about 70%, greater than about 75%, greater than about 80%, greater than about 85%, greater than about 90%, greater than about 95% or about 100% relative to the rest of the tissue.

### (b) Subject

A method of the invention may be used to detect or treat a tumor in a subject that is a human, a livestock animal, a companion animal, a lab animal, or a zoological animal. In one embodiment, the subject may be a rodent, e.g. a mouse, a rat, a guinea pig, etc. In another embodiment, the subject may be a livestock animal. Non-limiting examples of suitable livestock animals may include pigs, cows, horses, goats, sheep, llamas and alpacas. In yet another embodiment, the subject may be a companion animal. Non-limiting examples of companion animals may include pets such as dogs, cats, rabbits, and birds. In yet another embodiment, the subject may be a zoological animal. As used herein, a “zoological animal” refers to an animal that may be found in a zoo. Such animals may include non-human primates, large cats, wolves, and bears. In preferred embodiments, the animal is a laboratory animal. Non-limiting examples of a laboratory animal may include rodents, canines, felines, and non-human primates. In certain embodiments, the animal is a rodent. Non-limiting examples of rodents may include mice, rats, guinea pigs, etc.

### (c) Tumor

A composition of the invention may be used to treat or recognize a tumor derived from a neoplasm or a cancer, provided the tumor expresses cubilin. For example, the IF-B₁₂ conjugate administered intravenously binds cubilin outside the gastrointestinal tract. The IF-B₁₂ conjugate is then internalized and the detectable label and/or therapeutic agent is accumulated in cells expressing cubilin. By this mechanism, a composition of the invention may be used to treat or recognize a tumor. Cubilin is a large endocytic receptor serving such diverse functions as the intestinal absorption of the IF-B₁₂ complex and the renal proximal tubule reabsorption of filtered proteins including albumin, transferrin, vitamin D-binding protein and other important plasma carriers. Accordingly, cubilin is normally expressed in the intestine and the kidney. Importantly, cubilin overexpression has been observed in lung cancer and kidney cancer. As such, in an exemplary embodiment, methods of the invention may be used to detect or treat lung cancer and renal cell carcinoma. Additionally, methods of the invention may be used to detect or treat metastases associated with lung cancer and renal cell carcinoma. However, any other neoplasm that expresses cubilin may also be used in the methods of the invention.

“Neoplasm” is any tissue, or cell thereof, characterized by abnormal growth as a result of excessive cell division. The neoplasm may be malignant or benign, the cancer may be primary or metastatic; the neoplasm or cancer may be early stage or late stage. Non-limiting examples of neoplasms or cancers that may be treated or detected, provided they express cubilin, include acute lymphoblastic leukemia, acute myeloid leukemia, adrenocortical carcinoma, AIDS-related cancers, AIDS-related lymphoma, anal cancer, appendix cancer, astrocytomas (childhood cerebellar or cerebral), basal cell carcinoma, bile duct cancer, bladder cancer, bone cancer, brainstem glioma, brain tumors (cerebellar astrocytoma, cerebral astrocytoma/malignant glioma, ependymoma, medulloblastoma, supratentorial primitive neuroectodermal tumors, visual pathway and hypothalamic gliomas), breast cancer, bronchial adenomas/carcinoids, Burkitt lymphoma, carcinoid tumors (childhood, gastrointestinal), carcinoma of unknown primary, central nervous system lymphoma (primary), cerebellar astrocytoma, cerebral astrocytoma/malignant glioma, cervical cancer, childhood cancers, chronic lymphocytic leukemia, chronic myelogenous leukemia, chronic myeloproliferative disorders, colon cancer, cutaneous T-cell lymphoma, desmoplastic small round cell tumor, endometrial cancer, ependymoma, esophageal cancer, Ewing's sarcoma in the Ewing family of tumors, extracranial germ cell tumor (childhood), extragonadal germ cell tumor, extrahepatic bile duct cancer, eye cancers (intraocular melanoma, retinoblastoma), gallbladder cancer, gastric (stomach) cancer, gastrointestinal carcinoid tumor, gastrointestinal stromal tumor, germ cell tumors (childhood extracranial, extragonadal, ovarian), gestational trophoblastic tumor, gliomas (adult, childhood brain stem, childhood cerebral astrocytoma, childhood visual pathway and hypothalamic), gastric carcinoid, hairy cell leukemia, head and neck cancer, hepatocellular (liver) cancer, Hodgkin lymphoma, hypopharyngeal cancer, hypothalamic and visual pathway glioma (childhood), intraocular melanoma, islet cell carcinoma, Kaposi sarcoma, kidney cancer (renal cell cancer), laryngeal cancer, leukemias (acute lymphoblastic, acute myeloid, chronic lymphocytic, chronic myelogenous, hairy cell), lip and oral cavity cancer, liver cancer (primary), lung cancers (non-small cell, small cell), lymphomas (AIDS-related, Burkitt, cutaneous T-cell, Hodgkin, non-Hodgkin, primary central nervous system), macroglobulinemia (Waldenström), malignant fibrous histiocytoma of bone/osteosarcoma, medulloblastoma (childhood), melanoma, intraocular melanoma, Merkel cell carcinoma, mesotheliomas (adult malignant, childhood), metastatic squamous neck cancer with occult primary, mouth cancer, multiple endocrine neoplasia syndrome (childhood), multiple myeloma/plasma cell neoplasm, mycosis fungoides, myelodysplastic syndromes, myelodysplastic/myeloproliferative diseases, myelogenous leukemia (chronic), myeloid leukemias (adult acute, childhood acute), multiple myeloma, myeloproliferative disorders (chronic), nasal cavity and paranasal sinus cancer, nasopharyngeal carcinoma, neuroblastoma, non-Hodgkin lymphoma, non-small cell lung cancer, oral cancer, oropharyngeal cancer, osteosarcoma/malignant fibrous histiocytoma of bone, ovarian cancer, ovarian epithelial cancer (surface epithelial-stromal tumor), ovarian germ cell tumor, ovarian low malignant potential tumor, pancreatic cancer, pancreatic cancer (islet cell), paranasal sinus and nasal cavity cancer, parathyroid cancer, penile cancer, pharyngeal cancer, pheochromocytoma, pineal astrocytoma, pineal germinoma, pineoblastoma and supratentorial primitive neuroectodermal tumors (childhood), pituitary adenoma, plasma cell neoplasia, pleuropulmonary blastoma, primary central nervous system lymphoma, prostate cancer, rectal cancer, renal cell carcinoma (kidney cancer), renal pelvis and ureter transitional cell cancer, retinoblastoma, rhabdomyosarcoma (childhood), salivary gland cancer, sarcoma (Ewing family of tumors, Kaposi, soft tissue, uterine), Sézary syndrome, skin cancers (nonmelanoma, melanoma), skin carcinoma (Merkel cell), small cell lung cancer, small intestine cancer, soft tissue sarcoma, squamous cell carcinoma, squamous neck cancer with occult primary (metastatic), stomach cancer, supratentorial primitive neuroectodermal tumor (childhood), T-Cell lymphoma (cutaneous), testicular cancer, throat cancer, thymoma (childhood), thymoma and thymic carcinoma, thyroid cancer, thyroid cancer (childhood), transitional cell cancer of the renal pelvis and ureter, trophoblastic tumor (gestational), enknown primary site (adult, childhood), ureter and renal pelvis transitional cell cancer, urethral cancer, uterine cancer (endometrial), uterine sarcoma, vaginal cancer, visual pathway and hypothalamic glioma (childhood), vulvar cancer, Waldenström macroglobulinemia, and Wilms tumor (childhood). In a preferred embodiment, the cancer is selected from the group consisting of bladder carcinoma, breast carcinoma, cervical carcinoma, cholangiocarcinoma, colorectal carcinoma, esophageal carcinoma, gastric sarcoma, glioma, lung carcinoma, lymphoma, melanoma, multiple myeloma, osteosarcoma, ovarian carcinoma, pancreatic carcinoma, prostate carcinoma, stomach carcinoma, a head, a neck tumor, and a solid tumor.

### (d) Administration

In certain aspects, a pharmacologically effective amount of a composition of the invention may be administered to a subject. In another aspect, a pharmacologically effective amount of IF and a pharmacologically effective amount of B₁₂ or a B₁₂ conjugate are administered separately to a subject, such that IF and B₁₂ or a B₁₂ conjugate bind in vivo. Parenteral administration is performed using standard effective techniques. Parenteral administration includes but is not limited to subcutaneous, intravenous, intramuscular, intradermal, intra-arterial, intraosseous, intraperitoneal, or intrathecal injection, or infusion techniques. Effective parenteral systemic delivery by intravenous injection is a preferred method of administration to a subject. Suitable vehicles for such injections are straightforward.

Pharmaceutical compositions for effective administration are deliberately designed to be appropriate for the selected mode of administration, and pharmaceutically acceptable excipients such as compatible dispersing agents, buffers, surfactants, preservatives, solubilizing agents, isotonicity agents, stabilizing agents and the like are used as appropriate. Remington's Pharmaceutical Sciences, Mack Publishing Co., Easton Pa., 16Ed ISBN: 0-912734-04-3, latest edition, incorporated herein by reference in its entirety, provides a compendium of formulation techniques as are generally known to practitioners. It may be particularly useful to alter the solubility characteristics of the composition useful in this discovery, making it more lipophilic, for example, by encapsulating it in liposomes or by blocking polar groups.

For therapeutic applications, a therapeutically effective amount of a composition of the invention is administered to a subject. A “therapeutically effective amount” may be an amount of the therapeutic composition sufficient to produce a measurable biological tumor response (e.g., an immunostimulatory, an anti-angiogenic response, a cytotoxic response, or tumor regression). Alternatively, a “therapeutically effective amount” may be an amount of the therapeutic composition sufficient to produce a measurable decrease in cubilin function (e.g. albumin increase in the urine, prevention of acute kidney injury, treatment of acute kidney injury, prevention of uptake of nephrotoxic proteins such as light chains, myoglobin and hemoglobin, decrease of uptake of nephrotoxic proteins). Actual dosage levels of active ingredients in a therapeutic composition of the invention can be varied so as to administer an amount of the active compound(s) that is effective to achieve the desired therapeutic response for a particular subject. The selected dosage level will depend upon a variety of factors including the activity of the therapeutic composition, formulation, the route of administration, combination with other drugs or treatments, tumor size and longevity, and the physical condition and prior medical history of the subject being treated. In some embodiments, a minimal dose is administered, and dose is escalated in the absence of dose-limiting toxicity. Determination and adjustment of a therapeutically effective dose, as well as evaluation of when and how to make such adjustments, are known to those of ordinary skill in the art of medicine.

For diagnostic applications, a detectable amount of a composition of the invention is administered to a subject. A “detectable amount”, as used herein to refer to a diagnostic composition, refers to a dose of such a composition that the presence of the composition can be determined in vivo or in vitro. A detectable amount will vary according to a variety of factors, including but not limited to chemical features of the drug being labeled, the detectable label, labeling methods, the method of imaging and parameters related thereto, metabolism of the labeled drug in the subject, the stability of the label (e.g. the half-life of a radionuclide label), the time elapsed following administration of the drug and/or labeled peptide prior to imaging, the route of drug administration, the physical condition and prior medical history of the subject, and the size and longevity of the tumor or suspected tumor. Thus, a detectable amount can vary and can be tailored to a particular application. After study of the present disclosure, it is within the skill of one in the art to determine such a detectable amount.

A composition comprising IF and B₁₂ or B₁₂ conjugate may be administered at a concentration from about 0.1 pM to about 500 pM. For example, a composition comprising IF and B₁₂ or B₁₂ conjugate may be administered at a concentration of about 0.1 pM, about 0.2 pM, about 0.3 pM, about 0.4 pM, about 0.5 pM, about 0.6 pM, about 0.7 pM, about 0.8 pM, about 0.9 pM, about 1 pM, about 1.5 pM, about 2 pM, about 2.5 pM, about 3 pM, about 3.5 pM, about 4 pM, about 4.5 pM, about 5 pM, about 5.5 pM, about 6 pM, about 6.5 pM, about 7 pM, about 7.5 pM, about 8 pM, about 8.5 pM, about 9 pM, about 9.5 pM or about 10 pM. Alternatively, a composition comprising IF and B₁₂ or B₁₂ conjugate may be administered at a concentration of about 15 pM, about 20 pM, about 25 pM, about 30 pM, about 35 pM, about 40 pM, about 45 pM, about 50 pM, about 55 pM, about 60 pM, about 65 pM, about 70 pM, about 75 pM, about 80 pM, about 85 pM, about 90 pM, about 95 pM, about 100 pM, about 150 pM, about 200 pM, about 250 pM, about 300 pM, about 350 pM, about 400 pM, about 450 pM, or about 500 pM. In a specific embodiment, a composition comprising IF and B₁₂ or B₁₂ conjugate may be administered at a concentration of about 1 pM. In another specific embodiment, a composition comprising IF and B₁₂ or B₁₂ conjugate may be administered at a concentration of about 4 pM. In still another specific embodiment, a composition comprising IF and B₁₂ or B₁₂ conjugate may be administered at a concentration from about 1 pM to about 10 pM. In still yet another specific embodiment, a composition comprising IF and B₁₂ or B₁₂ conjugate may be administered at a concentration from about 10 pM to about 50 pM. In other embodiments, a composition comprising IF and B₁₂ or B₁₂ conjugate may be administered at a concentration from about 50 pM to about 500 pM.

In an embodiment where IF and B₁₂ or B₁₂ conjugate are administered separately, IF and B₁₂ or B₁₂ conjugate may be administered at the same or different concentrations. IF and/or B₁₂ or B₁₂ conjugate may be administered at a concentration from about 0.1 pM to about 500 pM. For example, IF and/or B₁₂ or B₁₂ conjugate may be administered at a concentration of about 0.1 pM, about 0.2 pM, about 0.3 pM, about 0.4 pM, about 0.5 pM, about 0.6 pM, about 0.7 pM, about 0.8 pM, about 0.9 pM, about 1 pM, about 1.5 pM, about 2 pM, about 2.5 pM, about 3 pM, about 3.5 pM, about 4 pM, about 4.5 pM, about 5 pM, about 5.5 pM, about 6 pM, about 6.5 pM, about 7 pM, about 7.5 pM, about 8 pM, about 8.5 pM, about 9 pM, about 9.5 pM or about 10 pM. Alternatively, IF and/or B₁₂ or B₁₂ conjugate may be administered at a concentration of about 15 pM, about 20 pM, about 25 pM, about 30 pM, about 35 pM, about 40 pM, about 45 pM, about 50 pM, about 55 pM, about 60 pM, about 65 pM, about 70 pM, about 75 pM, about 80 pM, about 85 pM, about 90 pM, about 95 pM, about 100 pM, about 150 pM, about 200 pM, about 250 pM, about 300 pM, about 350 pM, about 400 pM, about 450 pM, or about 500 pM. In a specific embodiment, IF and/or B₁₂ or B₁₂ conjugate may be administered at a concentration of about 1 pM. In another specific embodiment, IF and/or B₁₂ or B₁₂ conjugate may be administered at a concentration of about 4 pM. In still another specific embodiment, IF and/or B₁₂ or B₁₂ conjugate may be administered at a concentration from about 1 pM to about 10 pM. In still yet another specific embodiment, IF and/or B₁₂ or B₁₂ conjugate may be administered at a concentration from about 10 pM to about 50 pM. In other embodiments, IF and/or B₁₂ or B₁₂ conjugate may be administered at a concentration from about 50 pM to about 500 pM. In different embodiments, an excess of B₁₂ or B₁₂ conjugate relative to IF is administered.

Typical dosage levels can and will vary and may be determined and optimized using standard clinical techniques and will be dependent in part on the detectable label and/or therapeutic agent utilized and on the mode of administration.

The frequency of dosing may be daily or once, twice, three times or more per day, per week or per month, as needed as to effectively treat the symptoms. The timing of administration of the treatment relative to the disease itself and duration of treatment will be determined by the circumstances surrounding the case. Treatment could begin immediately, such as at the site of the injury as administered by emergency medical personnel. Treatment could begin in a hospital or clinic itself, or at a later time after discharge from the hospital or after being seen in an outpatient clinic. Duration of treatment could range from a single dose administered on a one-time basis to a life-long course of therapeutic treatments.

Although the foregoing methods appear the most convenient and most appropriate and effective for administration of the composition, by suitable adaptation, other effective techniques for administration may be employed provided proper formulation is utilized herein.

In addition, it may be desirable to employ controlled release formulations using biodegradable films and matrices, or osmotic mini-pumps, or delivery systems based on dextran beads, alginate, or collagen.

### (e) Optional Administration

In an aspect, the method may further comprise administration of free B₁₂. Although the transfer of conjugated B₁₂ between IF and TCII is likely to be limited, the administration of free B₁₂ may saturate TCII thereby preventing any low level transfer of conjugated B₁₂ from IF to TCII. Administration of B₁₂ is standard in the art. Typically B₁₂ is administered orally, subcutaneously, intramuscularly or intravenously. B₁₂ may be administered at a dose from about 1 to 2000 μg. The dose administered may be dependent on the route of administration and history of the subject. By way of non-limiting example, 1000 μg may be given intravenously or intramuscularly.

Administration of a composition to a subject can be performed by administering free B₁₂ prior to, concurrent with, or subsequent to administration of a composition of the invention. Accordingly, the free B₁₂ is administered in some embodiments 0 hours to about 24 hours before administration of a composition or formulation of the invention, and in some embodiments about 0 hours to about 4 hours before administration of a composition or formulation of the invention.

In another aspect, the method may further comprise administration of L-lysine. High renal uptake decreases imaging sensitivity in the abdomen. Administration of L-lysine is standard in the art and may be used to block renal uptake. The administration of L-lysine may be used to reduce background due to kidney uptake and may enhance visualization of metastases. For example, in an embodiment where RCC is suspected, L-lysine may be administered to block binding of a composition of the invention to renal cells of the kidney while still enabling detection of RCC metastases. Typically L-lysine is administered orally or intravenously. L-lysine may be administered at a dose from about 100 mg/kg to about 1000 mg/kg or from about 200 mg/kg to about 600 mg/kg.

Administration of a composition to a subject can be performed by administering L-lysine prior to, concurrent with, or subsequent to administration of a composition of the invention. Accordingly, the L-lysine is administered in some embodiments 0 hours to about 24 hours before administration of the composition, in some embodiments about 0 hours to about 4 hours before administration of the composition, and in some embodiments from about 30 min to 1 hour before administration of the composition.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Intrinsic Factor (IF)—B₁₂ Imaging Probe Rationale and Development

Vitamin B₁₂, also referred to as cobalamin, is an essential cofactor for many metabolic processes. FIG. 1 depicts the structure of B₁₂. The Co (III) atom is coordinated to a corrin ring, a ring that, in this case, has seven amide side chains (a-g).⁶ Within the planar corrin ring, there are four reduced pyrrole rings (depicted as A-D).⁶ The corrin ring experiences π delocalization between the nitrogen and sp² carbon atoms.⁶ Cobalamins with the cobalt atom in the +3 state usually have octahedral geometry with the ligand in the X position in the base-on form, meaning the nitrogen in the dimethylbenzimidazole group is coordinated to cobalt.⁶

B₁₂ enters the body in the mouth, where it is released from the food and bound by haptocorrin (HC), a ˜58 kDa protein (K_{d}=0.01 pM).⁶ The HC-B₁₂ complex then travels to the stomach, where gastric parietal cells release intrinsic factor (IF). As HC-B₁₂ enters the small intestine, HC partially unfolds due to an increase in pH and is then degraded by proteases, releasing B₁₂. In the blood, HC binds approximately 75% of cobalamin, while transcobalamin binds the remainder.⁹

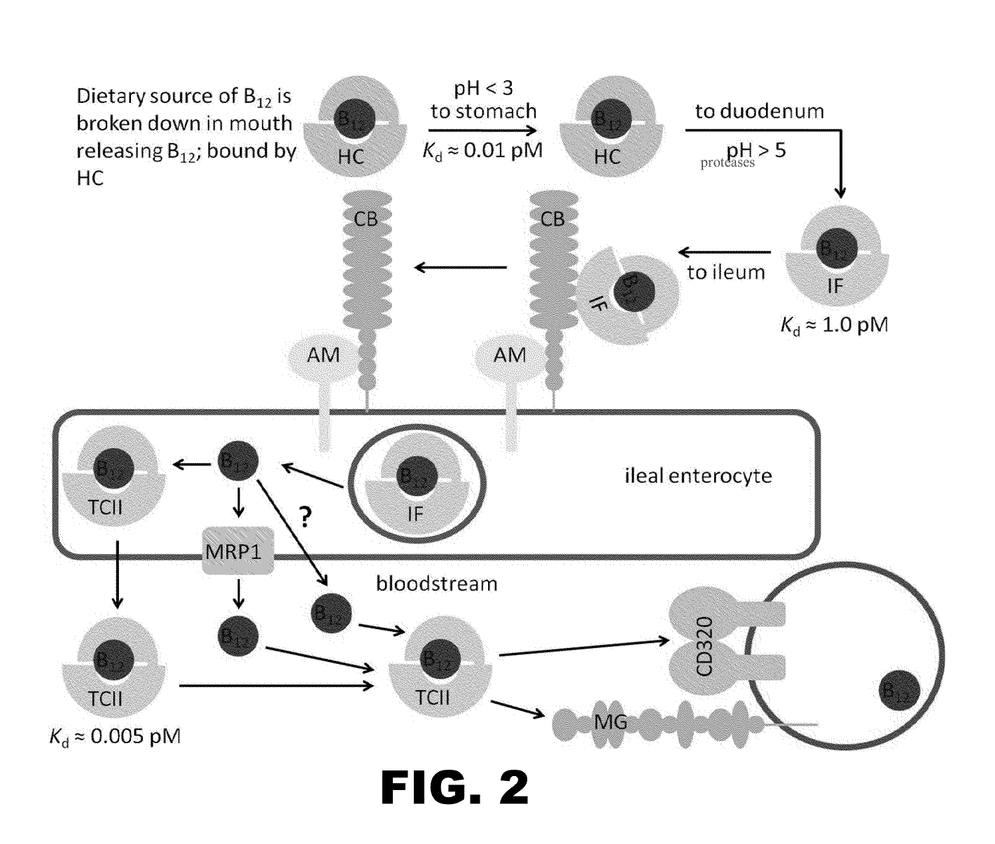

IF, a 50 kDa protein, binds B₁₂ (K_{d}=0.003 pM) when it is released from the degraded HC.⁶ IF-B₁₂ then travels to the duodenum where it binds to cubilin (CUBN) with high affinity (K_{d}˜5 nm). CUBN is a 460 kDa glycosylated peripheral membrane protein expressed on the surface of apical ileal enterocytes.⁷ Specifically, IF-B₁₂ binds to CUB domains 5-8 in a Ca²⁺ dependent manner.⁷ CUBN has a total of 27 CUB domains, which along with IF-B₁₂ bind albumin, transferrin, and vitamin D (bound to D binding protein).¹⁰ Due to the fact that CUBN has no transmembrane or cytoplasmic domains, in order for endocytosis to be possible two other transmembrane proteins, Amnionless (AMN) and Megalin/LRP2 must be present.⁷ Once inside the ileal enterocytes, IF is degraded by lysosomes and B₁₂ is bound by transcobalamin II (TCII) (˜44 kDa, K_{d}=0.004 pM).⁶ FIG. 2 depicts the dietary uptake pathway of B₁₂.

Due to the fact that cobalamin is necessary for cell proliferating processes and also has beneficial properties such as being water-soluble and having no known cytotoxicity, there are clear implications for the use of this vitamin as an imaging probe. Based on crystal structure studies of the binding of B₁₂ by haptocorrin (HC) and intrinsic factor (IF), there are positions on B₁₂ which are most ideal for modification that avoid obstructing natural biological processes. Conjugations can occur on the carboxylic acids that result from acid hydrolysis of the amide side chains, or the 5′-hydroxyl residue on the ribosyl group.⁶

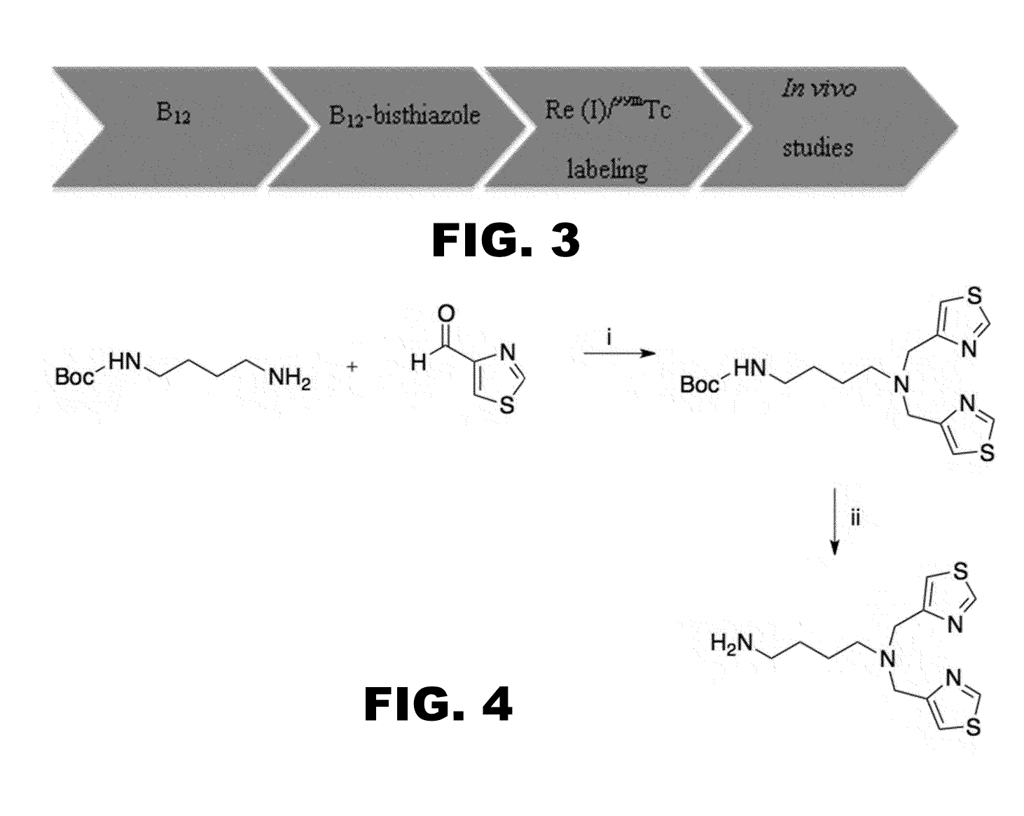

While imaging technology has advanced immensely, one of the biggest weaknesses of current imaging probes is the level of background emissions. Background is a term used to describe any areas in a specimen that are illuminated when imaged, but are not the preferential target. B₁₂ has a tendency to accumulate in the kidney and liver, thus skewing imaging results. In essence, the background found with B₁₂ probes stems from the prevalence of TCII receptors (MG, CD320) throughout the body, as all cells need B₁₂ for cell proliferation. In order to circumvent this problem, we designed an IF-B₁₂ probe that will target cubilin receptors, as opposed to TCII. Currently, the most studies cells with cubilin receptors are ileal enterocyte cells of the duodenum and kidney cells.⁹ It has also been shown that kidney cell receptors can be blocked by L-lysine co-infusion, therefore allowing for only metastatic tumors to be imaged as opposed to a large background from the kidney.¹¹ FIG. 3 depicts the schematic for synthesizing this probe.

### Example 2

### Synthesis of 1,1-bisthiazolate-(1,4)-diaminobutane (1)

1,1-bisthiazolate-(1,4)-diaminobutane (1) was synthesized by reacting N-Boc-1,4-butanediamine (300 mg, 1.5 mmol) with thiazole-4-carboxaldehyde (3 mmol, 300 mg) in anhydrous dichloroethane (DCE) at room temperature under argon for thirty minutes.¹² After this initial step, 5 mL of DCE were added along with sodium-triacetoxyborohydride (3 mmol, 636 mg). This reaction then continued for 16 hours, at which time the solvent was removed under reduced pressure and the product was resuspended in 5 mL of 10% methanol, 10% trifluoroacetic acid in water. This reaction went for 3 hours at room temperature in order to remove the Boc protecting group. The solvent was then removed under reduced pressure and the product was resuspended in 10% methanol in water. This reaction had a 9% yield. FIG. 4 depicts the schematic of 1,1-bisthiazolate-(1,4)-diaminobutane reaction.

### Example 3

### Purification and Characterization of 1,1-bisthiazolate-(1,4)-diaminobutane (1)

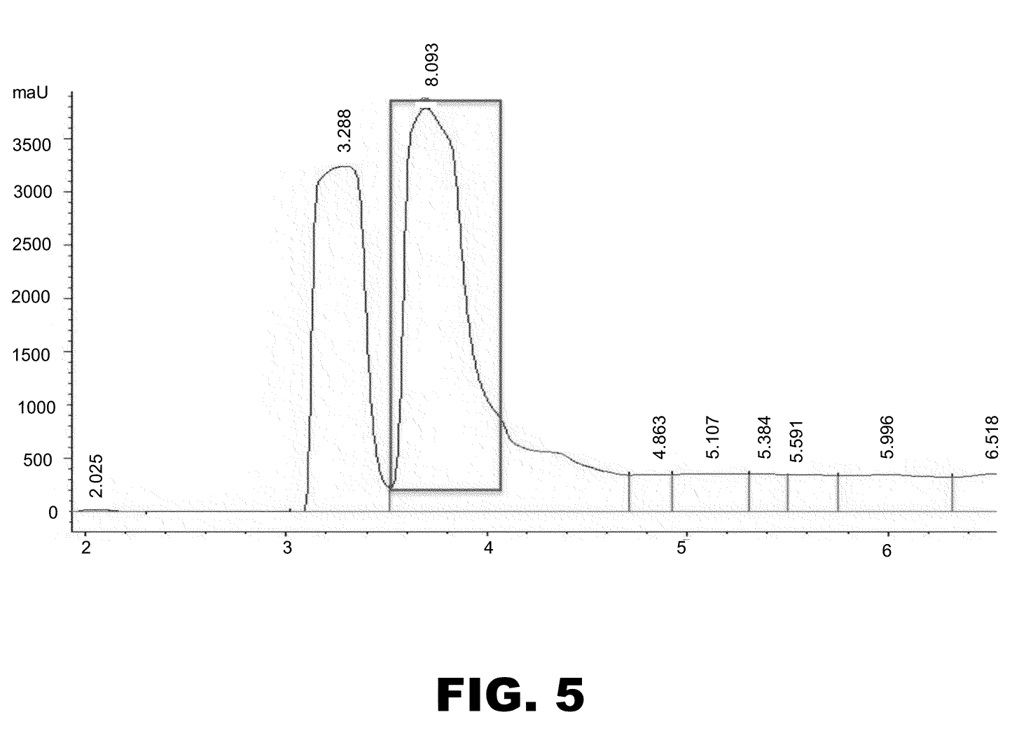

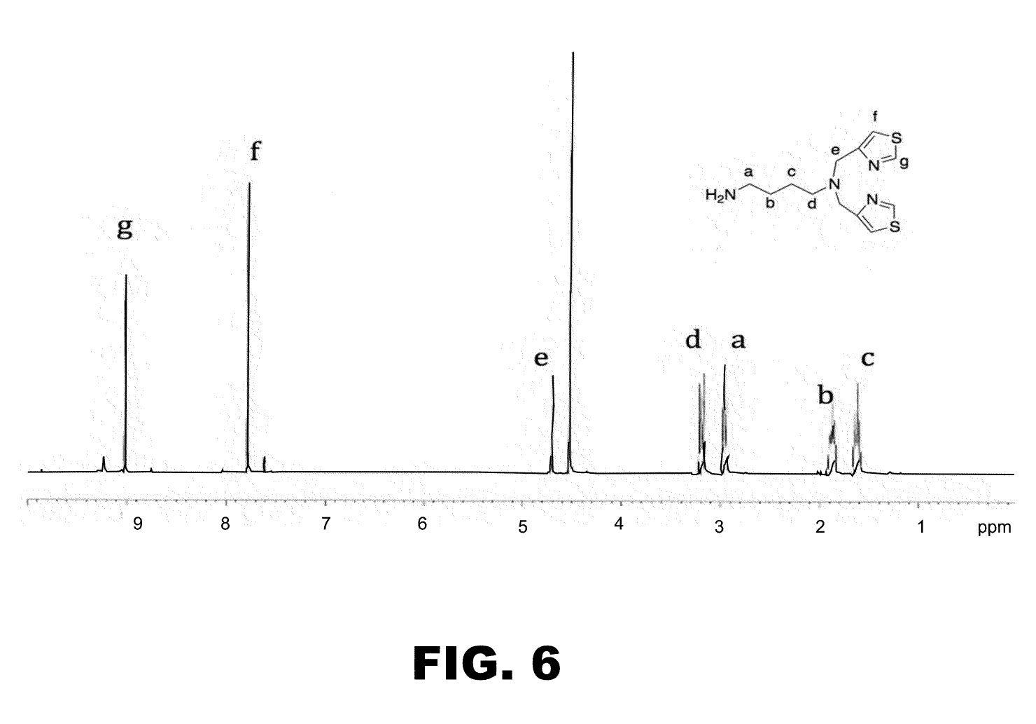

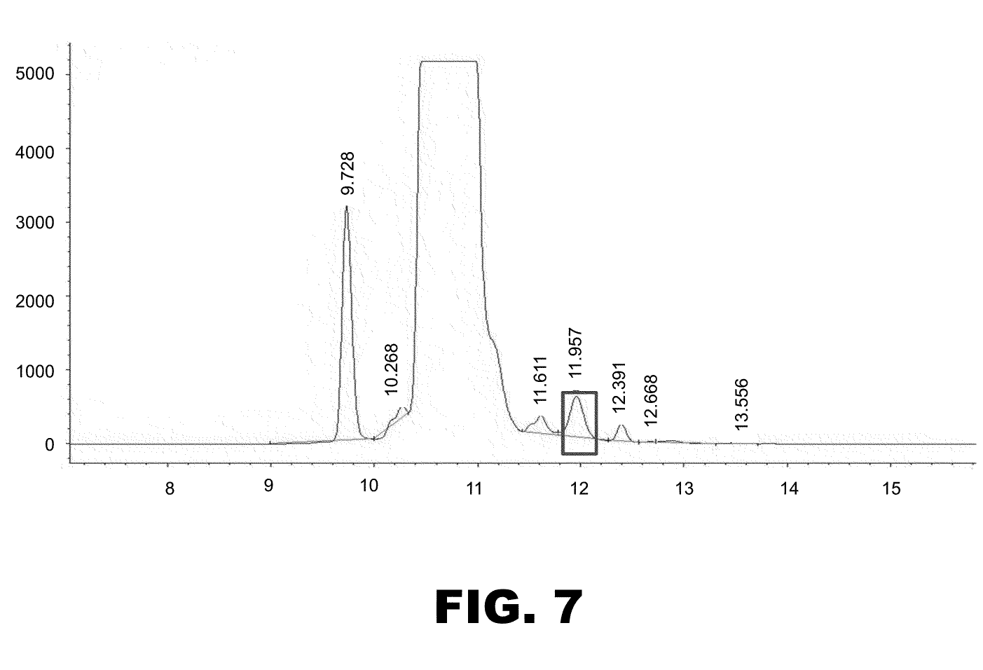

1,1-bisthiazolate-(1,4)-diaminobutane was purified using an Agilent 1200 series instrument with a quaternary pump at 254 nm UV detection on a C18 column (Agilent Eclipse XDB-C18). Solvent A was 0.1% TFA/H2O and solvent B was acetonitrile (MeCN). The gradient utilized to purify 1,1-bisthiazolate-(1,4)-diaminobutane was 0-5% MeCN over half a minute then hold at 5% MeCN for five minutes. FIG. 5 depicts the trace of this purification. ¹H-NMR was then performed to characterize 1,1-bisthiazolate-(1,4)-diaminobutane, as depicted in FIG. 6.

### Example 4

### Synthesis of B₁₂-bisthiazole (2)

0.145 mmol of Cyanocobalamin (196.53 mg) was activated by 0.725 mmol of 1,1′-Carbonyl-di-(1,2,4-triazole) (CDT) for 1 hour in 5 mL of dry DMSO at 50° C. 0.159 mmol (37.738 mg) of 1,1-bisthiazolate-(1,4)-diaminobutane in DMSO was added to the reaction, and allowed to react for 16 hours. The product was then precipitated using ether and acetone. The calculated yield of this reaction was 0.5%. In an attempt to increase reaction yield, this reaction was repeated using 0.045 mmol of cyanocobalamin (60.99 mg) activated by 0.36 mmol of CDT (59.34 mg) at 50° C. in dry DMSO for 1 hour and 15 minutes. The reaction then sat at room temperature for 15 minutes to cool down. 0.050 mmol of 1,1-bisthiazolate-(1,4)-diaminobutane (14 mg) in 1 mL of dry DMSO was added to the reaction mixture and reacted for 16 hours. The yield of this reaction was 1.5%. The B₁₂-bisthiazole reaction may be optimized to increase the yield

### Example 5

### Purification and Characterization of B₁₂-bisthiazole

B₁₂-bisthiazole was purified on an Agilent 1100 series instrument with a quaternary pump at 360 nm UV detection on a C18 column (Agilent Eclipse XDB-C18) with the gradient 0-5% MeCN over ten minutes, then 5-20% MeCN over 6 minutes. Solvent A was 0.1% TFA/H₂O and solvent B was acetonitrile (MeCN). FIG. 7 shows the RP-HPLC trace of B₁₂ Bisthiazole.

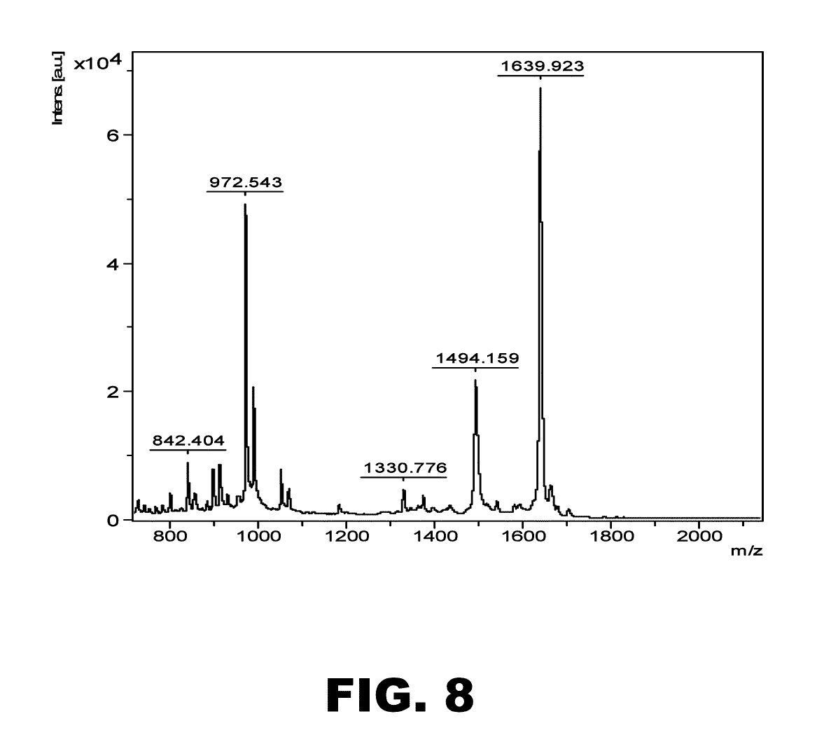

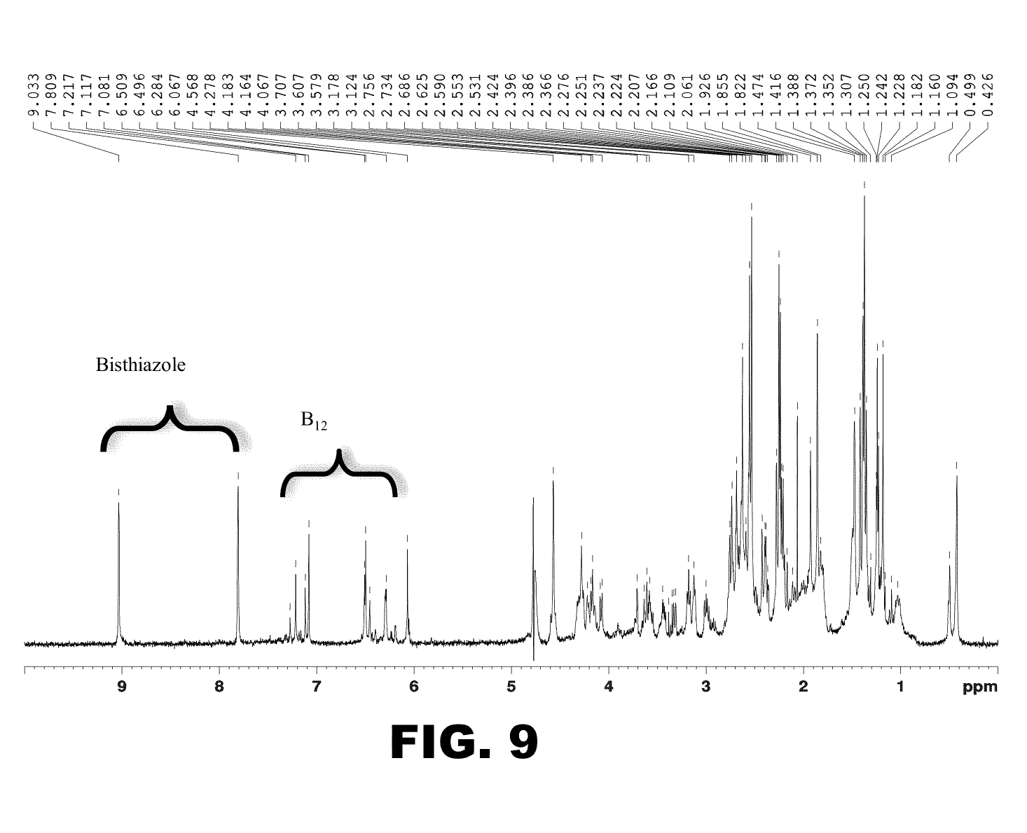

The purified product was characterized using matrix-assisted laser desorption ionization time of flight (MALDI-ToF). FIG. 8 shows the data representing the product at the m/z of 1907.615. B₁₂-bisthiazole was then lyophilized, resuspended in 500 μl of D₂O and characterized on a 400 MHz ¹H-NMR, shown in FIG. 9.

### Example 6

### Rhenium labeled B₁₂-bisthiazole as a Preliminary Probe Model

There are two isotopes of rhenium, ¹⁸⁸Re and ¹⁸⁶Re, which can be utilized for imaging purposes. Both isotopes are β⁻-emitters, however their emission properties are different. ¹⁸⁸Re is obtained from a generator that applies the beta decay of ¹⁸⁸W (t_{1/2}=62 days) and has a t_{1/2} of 16.2 hours and an Eₘₐₓ of 2.1 MeV.¹³ However, ¹⁸⁶Re has a t_{1/2} of 89.2 hours and an Eₘₐₓ of 1.1 MeV.¹³

Re(H₂O)₃(CO)₃Br had been previously synthesized by dissolving Re(CO)₅Br in dH₂O and refluxing at >100° C. for 24 h.⁹ The solution was then cooled, and filtered through a Celite plug.⁹ B₁₂-bisthiazole (2) (0.1 mg, 6×10⁻⁸ mol) and Re(H₂O)₃(CO)₃Br (0.03 mg, 6×10⁻⁸ mol) were refluxed in methanol at 60° C. for 3 hours.⁹ After the reaction was complete, the solvent was removed under reduced pressure and redissolved in 10% MeOH in water. The yield of this reaction was calculated to be 34%. The labeling of B₁₂-bisthiazole with Re(I) may be optimized to increase yield.

### Example 7

### Purification and Characterization of B₁₂-bisthiazole-Re(I) (2′

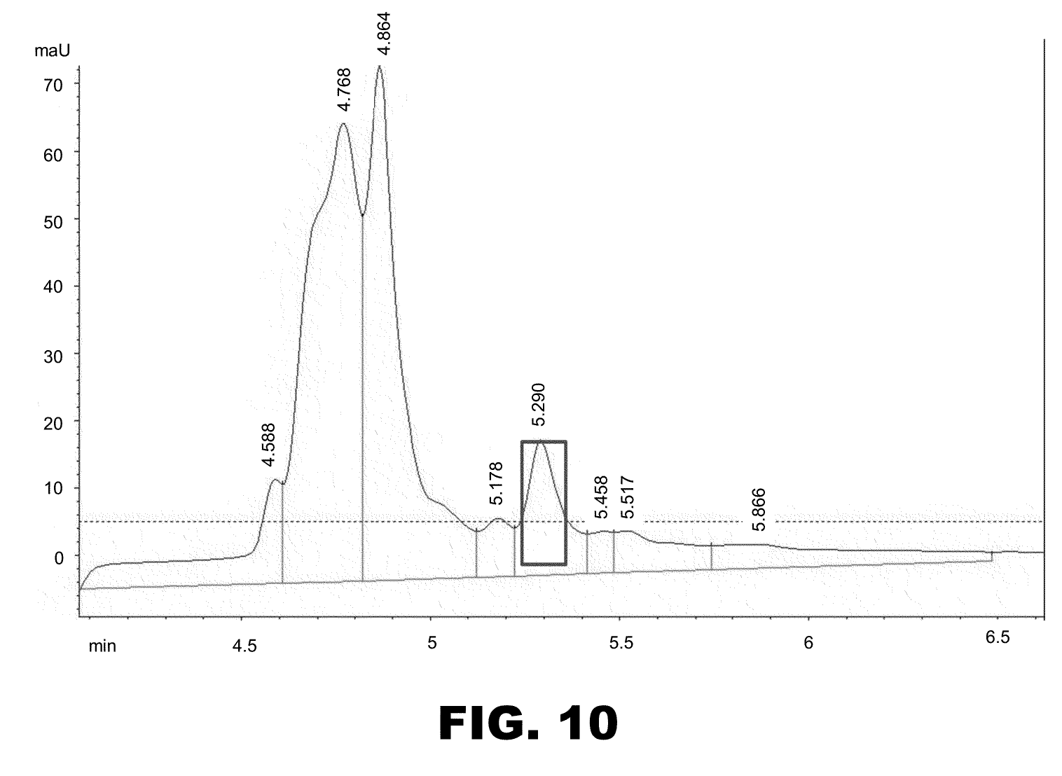

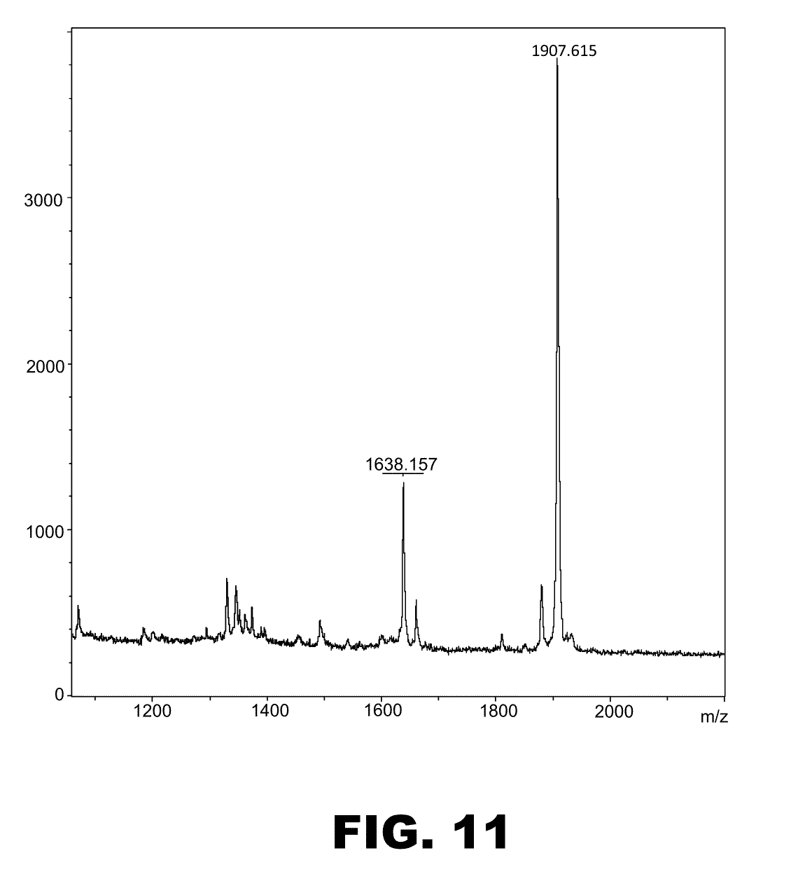

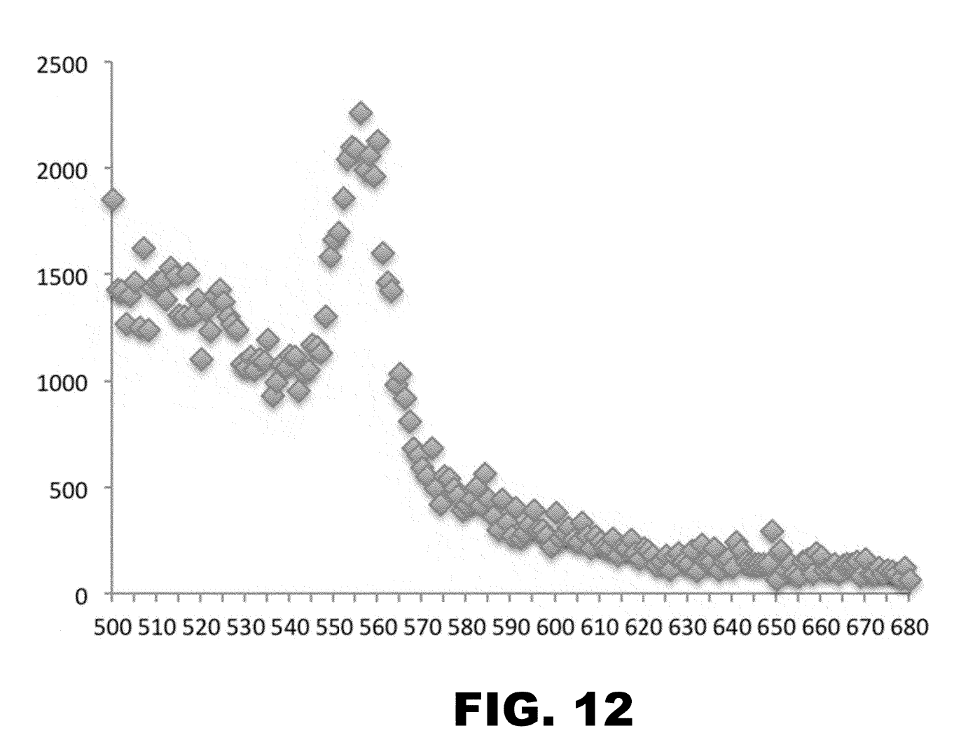

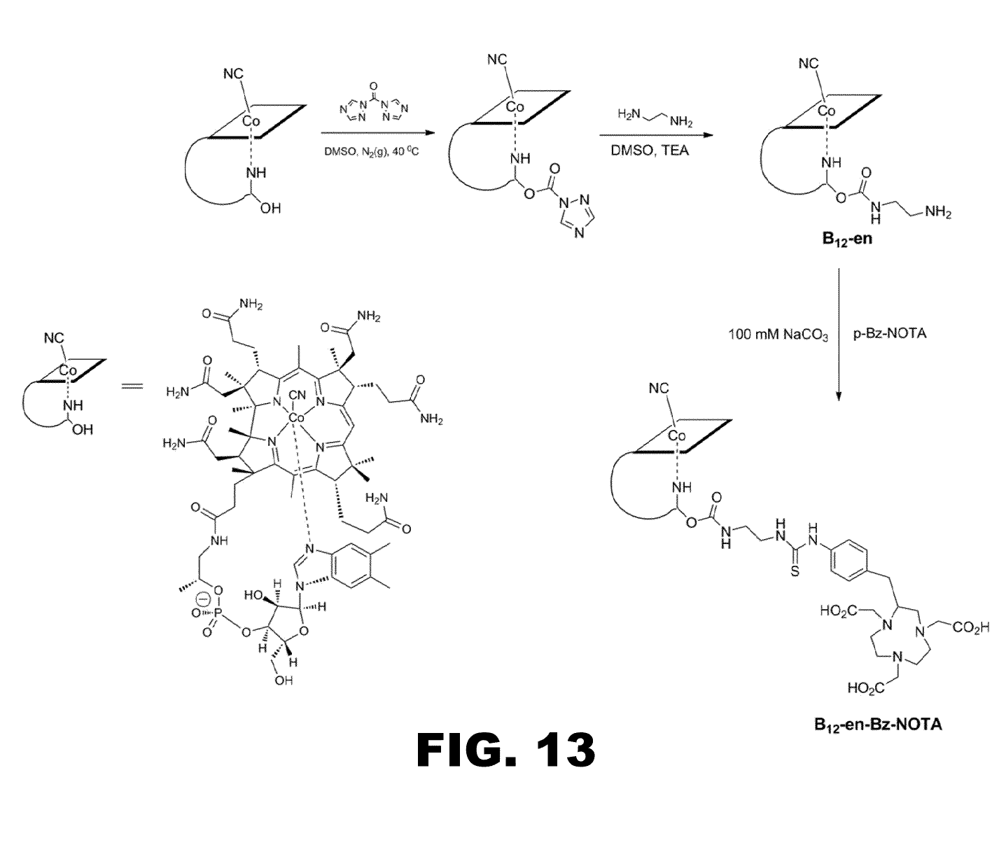

B₁₂-bisthiazole-Re(I) (2′) was purified using HPLC on Agilent 1100 series instrument with a quaternary pump at 360 nm UV detection on a C18 column (Agilent Eclipse XDB-C18). Solvent A was 0.1% TFA/H₂O and solvent B was acetonitrile. The method used was 0-15% B over five minutes and then 15-20% B over five minutes. FIG. 10 depicts the RP-HPLC trace with the Tᵣ of B₁₂-bisthiazole-Re(I) at 5.28 minutes. B₁₂-bisthiazole-Re(I) was then characterized using MALDI-ToF. The expected m/z was 1907 and the found m/z was 1907.615. FIG. 11 depicts the MALDI-ToF spectra of B₁₂-bisthiazole-Re(I). Finally, a fluorescence scan was performed on B₁₂-bisthiazole-Re(I) to provide evidence that it was the Re(I) that resulted in the changed mass, and that Re(I) was active. FIG. 12 depicts the fluorescence scan trace. In vivo studies in rats may be conducted to observe the tissue distribution of this imaging probe.

### Example 8

### ^{99m}Tc as a Radiometal

^{99m}Tc is formed through the decay of ⁹⁹Mo, which is a β⁻-emitter with a t_{1/2} of 66 hours.¹³ Due to differences in charge of these two products, they can easily be separated on an alumina chromatography column.^{13 99m}Tc is a γ-emitter, making it applicable to imaging that utilizes a gamma camera.¹³ Because the transition state from ^{99m}Tc to its ground state is nuclear-spin-forbidden, the half-life of ^{99m}Tc is relatively short at 6 hours, an optimum time for imaging in patients.¹³ The γ-energy is 140 keV, an energy large enough to penetrate biological tissues but small enough to minimize high dose burden to patients.¹³ B₁₂-bisthiazole will be labeled with ^{99m}Tc. Then in vivo studies in rats will be conducted to observe the tissue distribution of this imaging probe.

### Example 9

### Vitamin B₁₂ Conjugate, B₁₂-En-Bz-NOTA Radiolabeled with ⁶⁴Cu

As seen in FIG. 13, a radiolabeled B₁₂ conjugate may comprise the vitamin B₁₂ conjugate, B₁₂-en-Bz-NOTA, that is radiolabeled with ⁶⁴Cu under acidic conditions (pH 5.5) while heating at 60° C. for 30 minutes. B₁₂-en-Bz-NOTA may be synthesized by covalently conjugating B₁₂ to ethylenediamine (en) via a two-step coupling reaction, where the 5′-OH group followed by functionalization with an amine group for chelator incorporation. The desired B₁₂-en may be separated from unreacted B₁₂ using cation-exchange chromatography. Unreacted B₁₂ may be eluted in the flowthrough while B₁₂-en was eluted at 32 min with 10 mM NaCl. The p-isothiocyanatobenzyl functionalized NOTA chelate may be reacted with B₁₂-en as 1:1 mole equivalents; and the desired product, B₁₂-en-Bz-NOTA, isolated on an Eclipse XDB-C18 column with a retention time of 14.2 min. MALDI-ToF MS may be used to confirm the identity of the product with a peak at 1867 m/z consistent with B₁₂-en-Bz-NOTA without the cyano group of B₁₂.

### Example 10

### Binding of Conjugated B₁₂ to IF

Once the radioisotope is selected and conjugated to B₁₂, IF may be pre-bound to the B₁₂ conjugate by combining the conjugate with IF in solution as IF has picomolar affinity for B₁₂ and thus will bind if placed in solution together. For example, the radiolabeled B₁₂ system may be combined with IF in PBS at pH 7.4 or in MES buffer at pH 5.5. The radiolabeled B₁₂ conjugate pre-bound to IF may be studied to evaluate the efficacy of imaging and/or treatment.

### Example 11

### In Vitro Studies to Determine the Retention of IF and B₁₂ Binding in the Presence of TCII and HC

Two in vitro studies may be used to establish the efficacy of the present invention. First, an IF vs TCII/HC B₁₂ binding assay may be performed using 3.3 pM of IF-B₁₂-AF647 added to 1 nM of TCII/HC. Second, 1 nM of IF-B₁₂-AF647 should be added to 1 nM of TCII/HC. After the B₁₂ binding proteins have had time to compete for the B₁₂-Cu⁶⁴ they will be sequestered separately from the media via immunoprecipitation. The isolation of each of the binding proteins will be performed using anti-human IF/TCII/HC antibodies bound to magnetic microspheres. Each protein will be pelleted and measured for gamma emission using a gamma counter. The concentration of B₁₂-Cu⁶⁴ bound to each of the proteins will be measured in counts per minute (cpm) and compared to a calibration of holo-IF/TCII/HC-B₁₂-Cu⁶⁴. This will allow for a determination of how much B₁₂-Cu⁶⁴ has been stripped from the IF-B₁₂-Cu⁶⁴.

3.3 μM of Cu⁶⁴, equaling approximately 3.7 MBq (100 μCi) is the lowest detectable level that can be observed with PET imaging. TCII and HC are present in the blood in pM concentration, so if they strip the B₁₂-Cu⁶⁴ from IF-B₁₂-Cu⁶⁴, the levels will be below a detectable limit, i.e., no background imaging via CD320 will be observed.

### Example 12

### In Vivo Studies to Determine Biodistribution of the B₁₂-IF Probe

The present invention may also be evaluated by in vivo studies. To reduce interference by exogenous B₁₂, animals may be fed a B₁₂ deficient diet for 2 weeks prior to inoculation.

In a first study, preferably in rats, the IF-B₁₂-Cu⁶⁴ conjugate will be administered to 3 rats via tail vein injection. At specific time points (2, 6 and 24 hours), the rats will be scanned via PET imaging to determine the uptake of the conjugate. The focus of this experiment is to observe the biodistribution of cubilin throughout the various tissues.

In a second study of competitive blocking, preferably in rats, a predetermined concentration of free B₁₂ will be pre/co-injected to saturate TCII and HC in the blood. IF-B₁₂-Cu⁶⁴ will then be administered via tail vein injection and PET imaging will be performed at 2, 6 and 24 hours. Biodistribution of the conjugate will be compared to the first rat study.

A third study of tumor uptake, preferably in rats, should be performed in triplicate, with groups A, B and C consisting of n rats. Group A will be implanted with a kidney tumor cell line, known to express cubilin. Group B will be implanted with a lung cancer cell line, known to express cubilin. Group C will be implanted with a cancer cell line known to not express cubilin.

The studies should demonstrate that IF-B₁₂-Cu⁶⁴ can locate metastasized kidney tumors that express cubilin. It is expected to yield a greater specificity in tumor imaging, with decreased background over previous techniques. Due to expression of cubilin in certain tissues such as the kidney and the intestine, uptake and accumulation in these tissues may result in background imaging. Accordingly, accumulation of the conjugate in the kidney may be blocked with a number of approaches. The most common approach, intravenous injection of the cationic amino acid lysine, has successfully been used in the past to block or greatly reduce kidney uptake. For example, pre injection of 50 mg of 1-lysine two minutes before an 1¹²⁵-labeled conjugate decreased kidney uptake by approximately 95% in mice. Regardless, one of the most common locations of a metastasized kidney tumor is in the lung so accumulation in the intestine should not block imaging in the upper abdominal area.

### Example 13

### Targeted B₁₂ Conjugates Using L-Propargyl Glycine

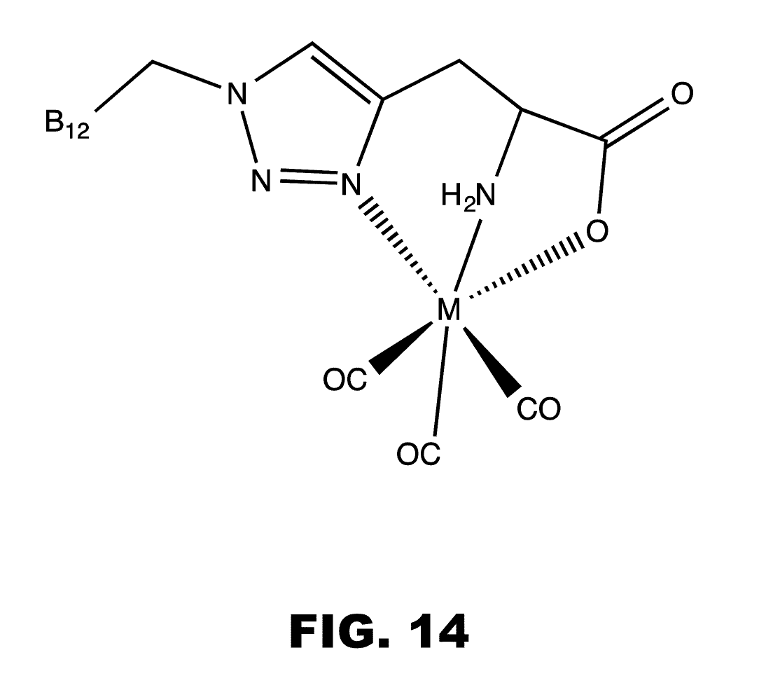

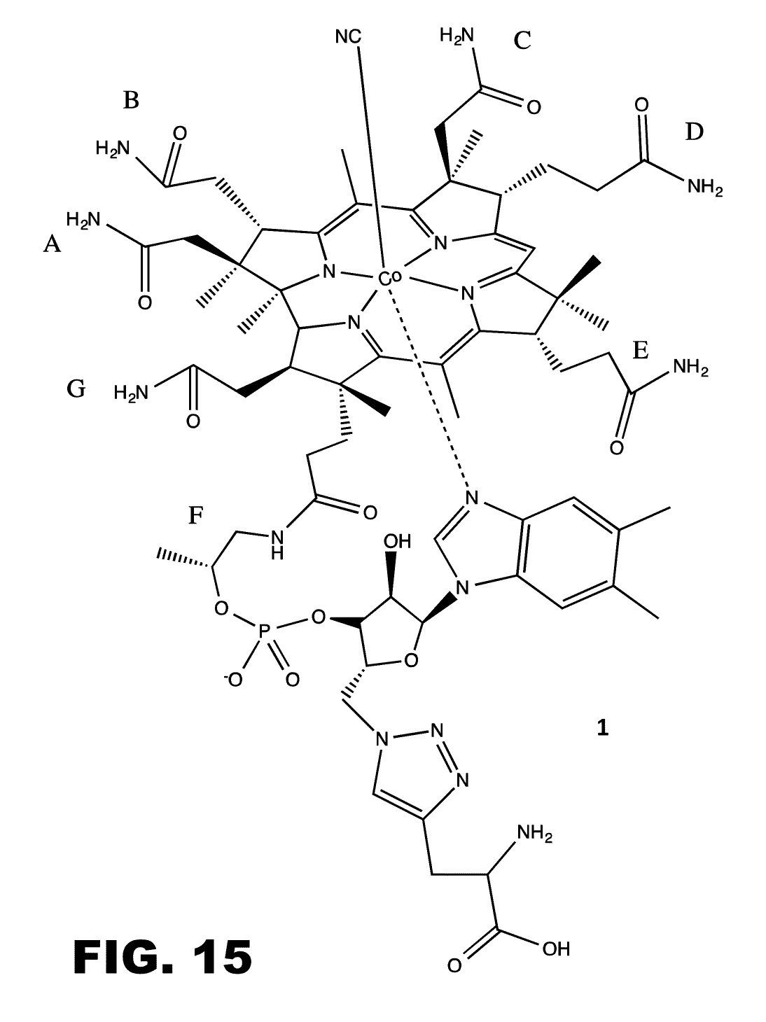

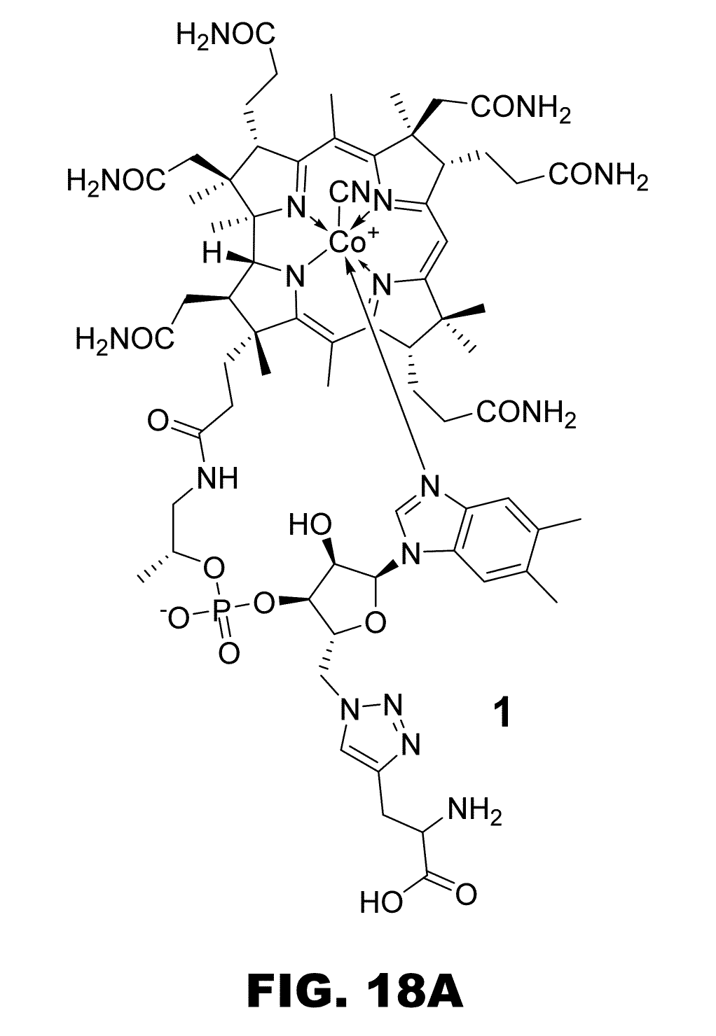

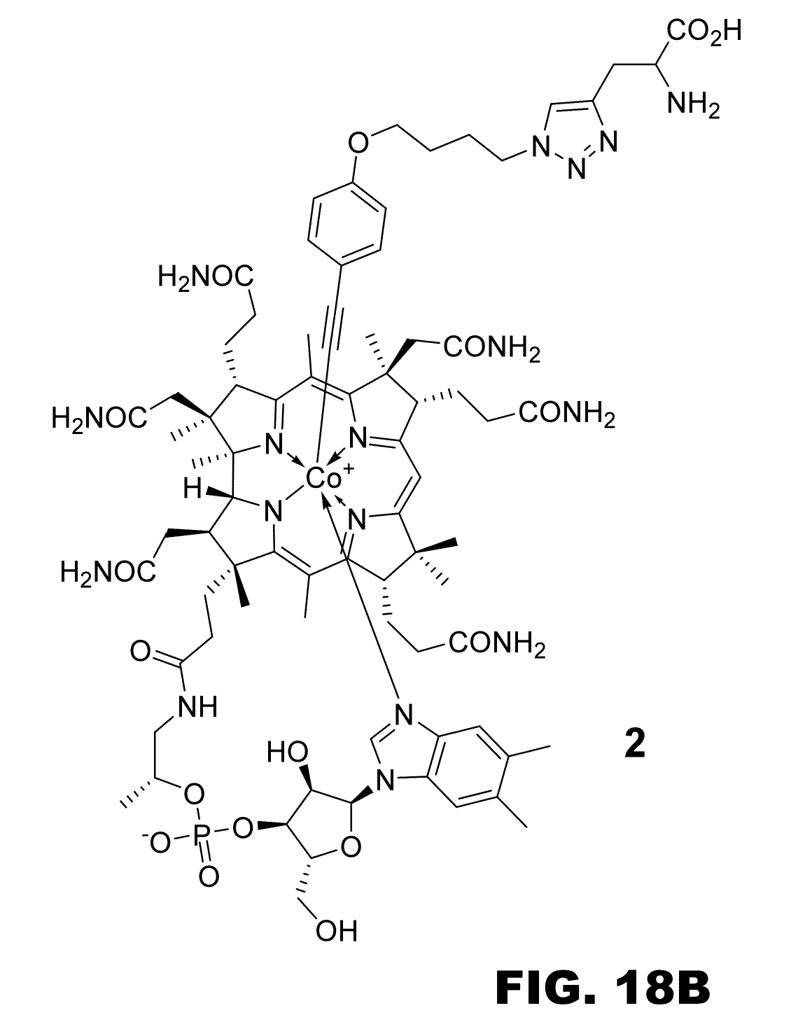

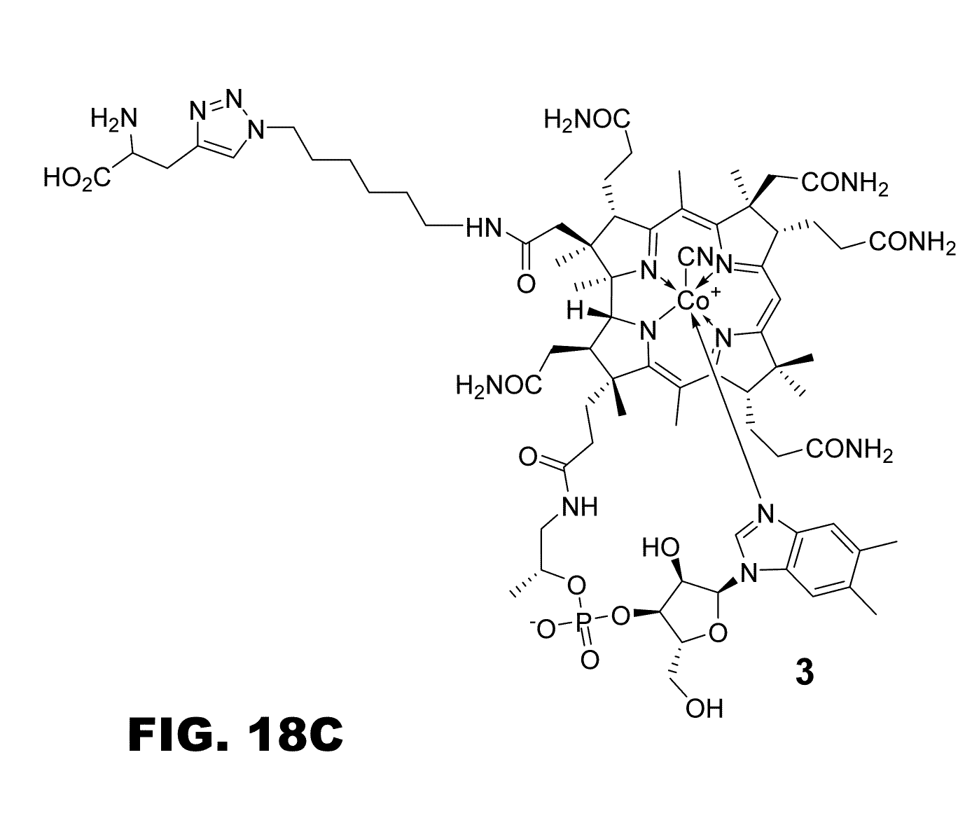

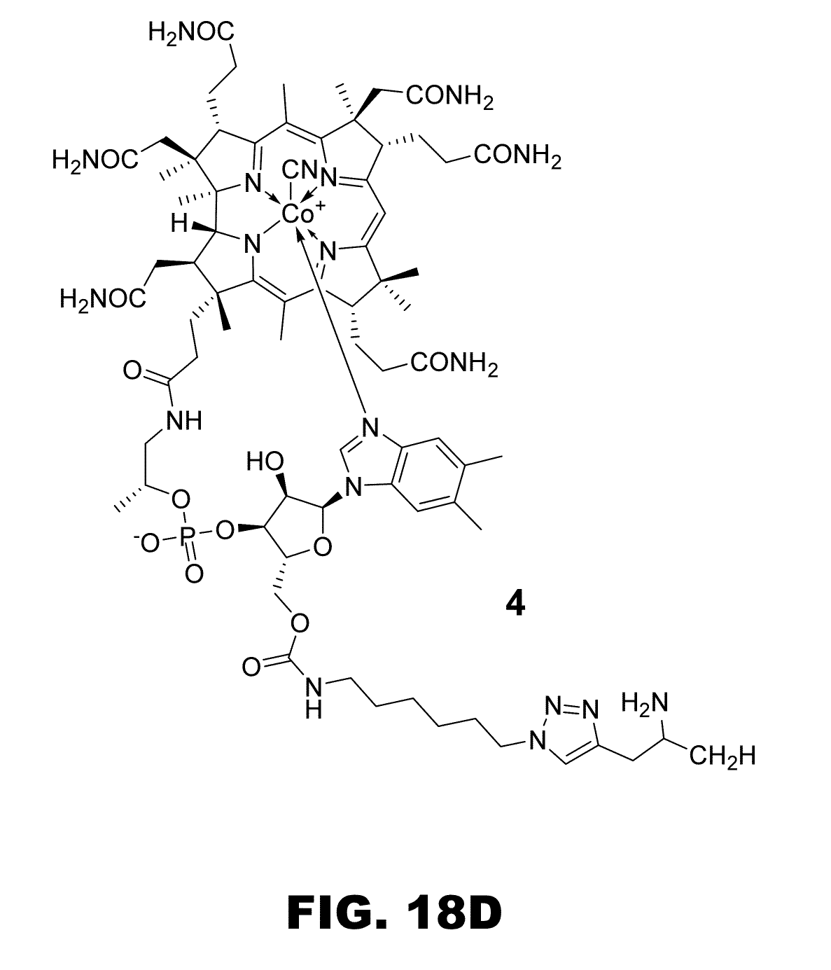

A B₁₂ conjugate may also be prepared using L-propargyl glycine. The L-propargyl glycine may then be chelated to a metal, such as ^{99m}Tc (FIG. 14). Notably, the L-conformation is important for metal binding. For example, the 5′-OH is transformed into a good leaving group and subsequently substituted with an azide. The resulting “clickable” azide is stable and highly active in the copper-catalyzed as well as in the strain promoted [1,3] dipolar cycloaddition (CuAAC or SPAAC) to alkynes. This methodology is described in detail in Chrominski et al, Chem Eur J2013; 19: 5141-5148. An alkyne containing glycine is then added using “click” chemistry (FIG. 15). Additionally, an alkyl chain linker may be added prior to the glycine as shown in 4 (FIG. 18D).

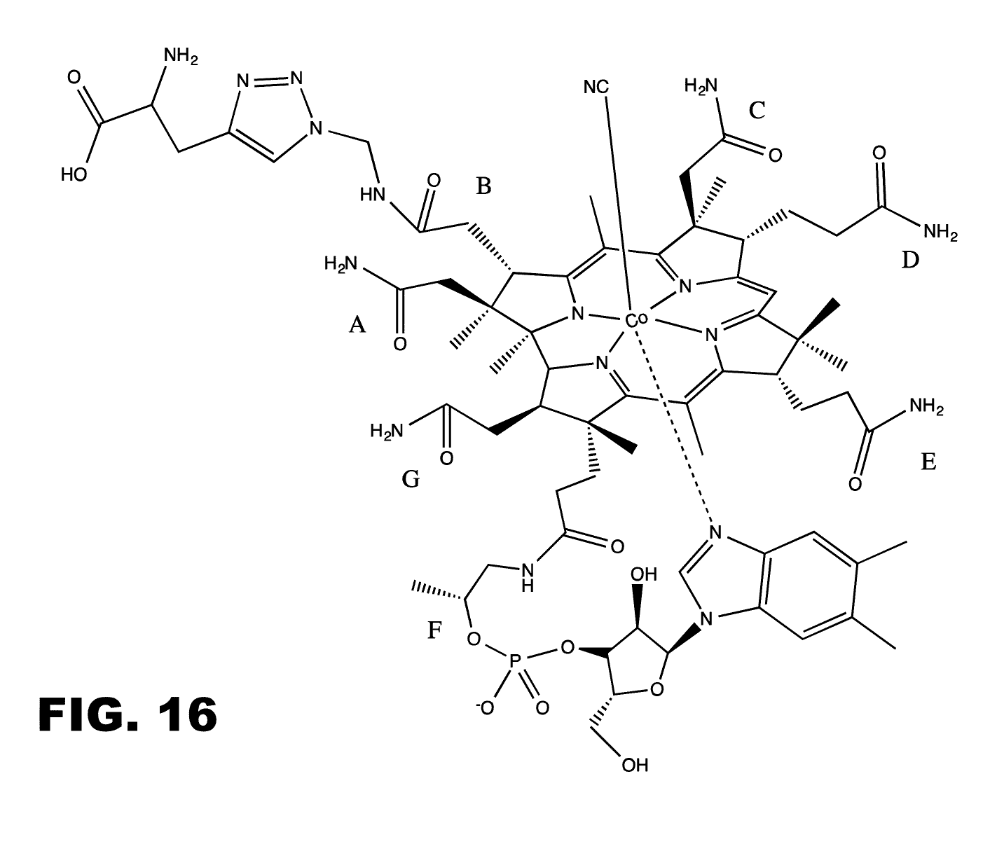

Alternatively, carefully controlled partial hydrolysis of cyanocobalamin under acidic conditions gives access to desirable b and e acids. For example, upon conversion to a carboxylic acid at the b-position, an alkyne containing glycine is added (FIG. 16). In an example, an alkyl chain linker is added prior to the glycine as shown in 3 (FIG. 18C).

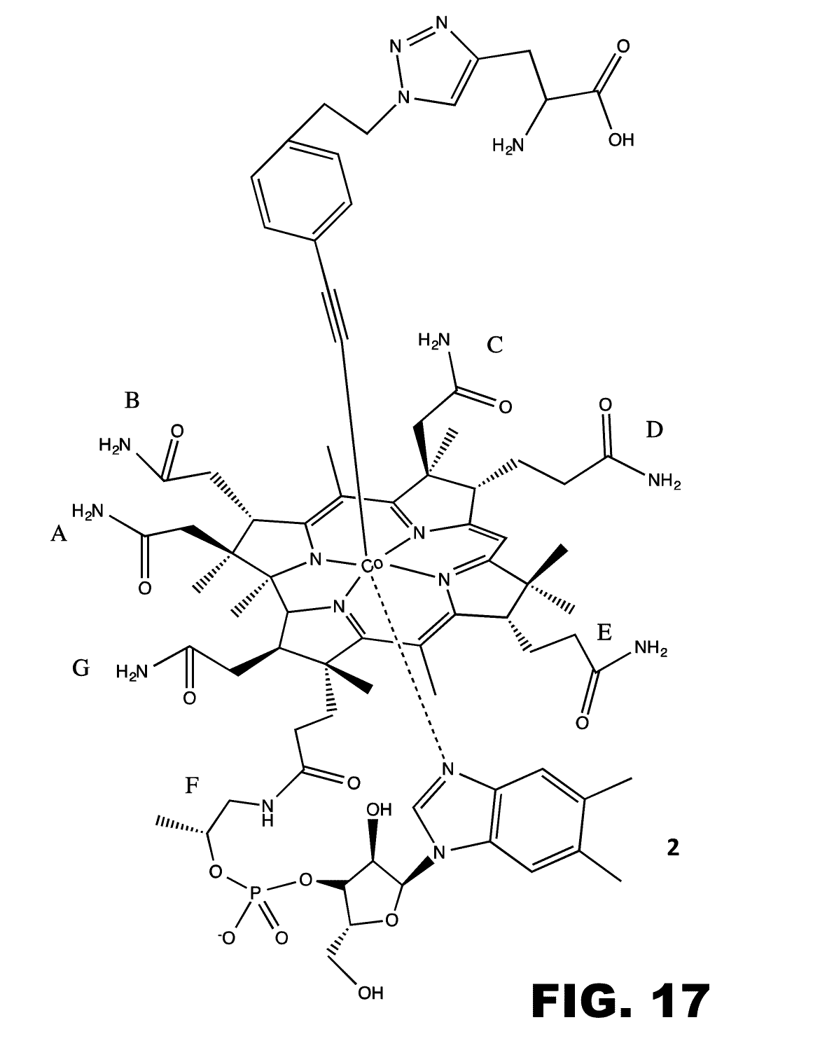

Yet another option is to conjugate B₁₂ using L-propargyl glycine at the cobalt ion resulting in a B12 conjugate with cobalt-C bond attachment. For example, direct reaction of (CN)CbI with a terminal alkyne in the presence of Cu(I) salts may furnish acetylides. The product may be further functionalized via [1,3] dipolar azide-alkyne cycloaddition. Specifically, L-propargyl glycine may be added with or with an alkyl chain linker (FIG. 17 and FIG. 18B).

### Example 14

### Receptor Mediated Uptake of Vitamin B₁₂-Conjugate Systems of >160 kDa

We wished to investigate and visualize in vitro the ability of cubilin to facilitate passage of a large protein. Size limitation of conjugates using the B₁₂ uptake pathway has also not been explored to date. Presented here is the successful in vitro uptake of B₁₂-tetanus toxoid conjugates (MW>160 kDa), through the cubilin receptor demonstrating the uptake pathway's potential to deliver large proteins.

B₁₂ was activated using CDT at 60° C. in dry DMSO. The activated B₁₂ was added in aliquots over one hour to the tetanus toxin (TT) in 50 mM carbonate buffer at pH 9.6. The TT is rich in lysine residues making CDT an ideal conjugation route. Two different amounts [1 mg (0.007 mmol) or 20 mg (0.0148 mmol)] of activated B₁₂ were reacted with 2400 L_{f} of TT to give conjugates 2 and 1, respectively. Purification of the conjugated system was achieved using gel permeation chromatography. The different synthetic ratios of B₁₂ gave similar chromatographic behavior, with the TT clearly dominating the separation behavior.

The weight of the TT itself was initially established and was noted at ˜158 kDa by mass spectrometry (MALDI-Tof). The additional 8 kDa on the predicted 150 kDa (based on amino acid sequence) most likely comes from the incorporation of formalin and lysine during the inactivation process. This established a baseline molecular weight and was used to compare to the new peaks from the HPLC separation. The mass spectrometry also showed free separate light and heavy chains in the TT sample.

The molecular weight of conjugate 1 was approximately 170 kDa. This is 12 kDa higher than the experimentally observed weight of the TT. The mass increase from the free TT to the conjugated TT is equivalent to nine B₁₂ molecules. By using a reaction with a smaller synthetic ratio of B₁₂ to TT, a molecular weight of 163 kDa was observed by mass spectrometry. For 2, compared to the free TT again, the shift in molecular weight was only 5 kDa. This is consistent with addition of four molecules of B₁₂.

To confirm the biological activity of the conjugate, an ELISA was developed. Rabbit polyclonal anti-TT antibodies were conjugated to 96 well plates. After incubating TT dilution standards (240-0.002 Lf/mL), different concentrations of mouse monoclonal anti-TT (1-0.5 μg/mL) were incubated to determine the appropriate dilution needed for a valid assay. Secondary antibodies conjugated to horseradish peroxidase were then incubated in the wells. The assay used a concentration range 1 L_{f}/ml to 0.005 L_{f}/mL that was found to generate a linear calibration curve. The calibration line however was only linear up to ˜10 L_{f}/mL concentrations of TT. In order to make sure that the conjugate's and the control's ELISA results are comparable, the concentration of protein in each sample was determined by Bradford assy.

Combining the Bradford results with the ELISA results, the samples are presented as ratio of L_{f} to μg. This number was compared to a Bradford assay done on the TT starting material, which gave an L_{f} to μg ratio of 0.32. The ratio of L_{f}/μg in the conjugates is lower than the TT starting material.

Both conjugates were then exposed to IF and binding was followed by electronic absorption spectroscopy as described previously. In both cases, IF binding was noted as indicated by an increase in absorption. Gel permeation chromatography was also consistent with IF-conjugate binding.

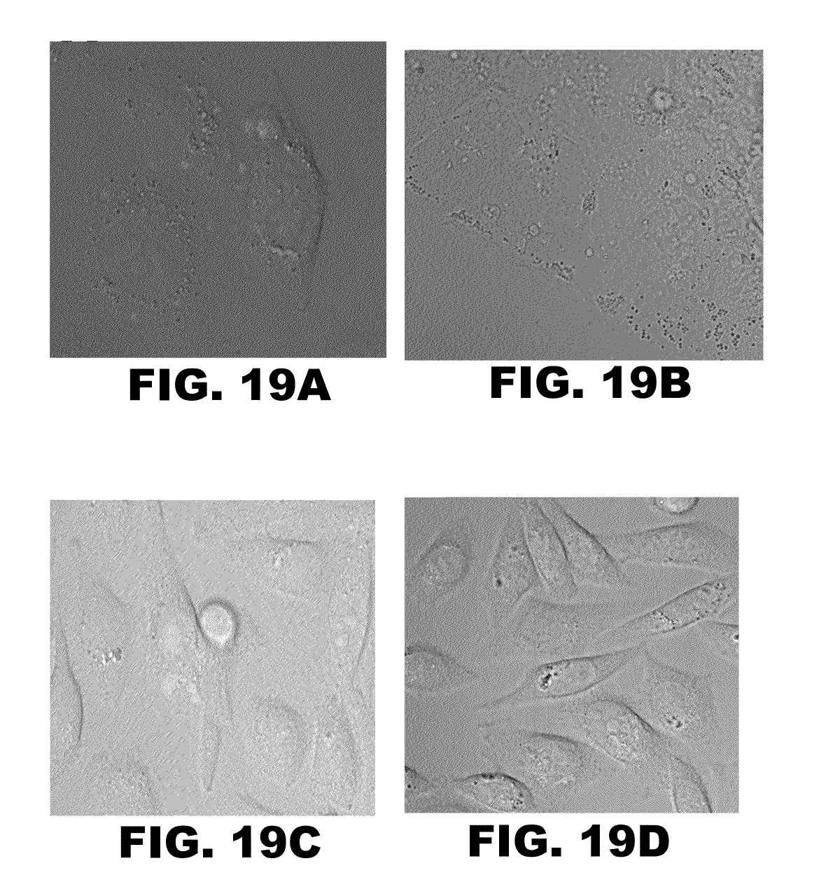

To establish a baseline for the presence of cubilin in the BeWo cell line, cubilin immunostaining was conducted. Antibodies to cubilin were tagged with Alexa Fluoro 405 dye (CubAb₄₀₅). The antibodies were then dialyzed for 24 hours to remove excess dye (followed by HPLC, not shown). The BeWo cells were incubated with CubAb₄₀₅ for 45 min at 37° C., and then examined with confocal microscopy (FIG. 19A). The cells show binding to the surface and some internalization of CubAb₄₀₅. The CubAb₄₀₅ was also incubated against the Chinese hamster ovary cell line (CHO). CHO cells do not express the cubilin receptor and therefore are a suitable negative control. The CubAb₄₀₅ did not show binding or uptake to the CHO cells (data not shown).

In order to rule out the possibility of TT mediated uptake in the BeWo cells, the TT was conjugated to the AlexaFluoro 405 tag (TT₄₀₅). TT₄₀₅ was incubated with the BeWo cell line with IF present. The BeWo cells did not take up the TT₄₀₅, to support the B₁₂ mediated uptake hypothesis. The TT₄₀₅ did show some slight membrane interaction but critically no internalization.

With confirmation that the cell line contains cubilin and that TT does not facilitate uptake, 1 and 2 were conjugated with CypHer 5E dye to make fluorescent conjugates (1_{C5E}, 2_{C5E}). CypHer 5E was chosen for these conjugates because under neutral conditions, the tag is fluorescently silent. When exposed to an acidic pH such as 5.5, it fluoresces. Observing fluorescence would suggest that the uptake is proceeding through receptor mediated endocytosis. 1_{C5E} was initially incubated with IF for 30 minutes, then incubated with BeWo cells for 1 hour. The cells were then washed with PBS at pH of 7.4 (×3) and at a pH of 3.0 (×1) to wash away free conjugate and reduce nonspecific membrane interactions. PBS at pH 7.4 was then added to the cells, and they were examined with confocal microscopy to look for uptake and internalization (see FIG. 19B). 1_{C5E} clearly demonstrates internalization in the cells.

To confirm an IF mediated uptake of the B₁₂ tetanus conjugate, a colocalization experiment was conducted with fluorescently-tagged IF (IF₄₀₅). IF was reacted with the Alexa Fluor 405 for one hour and then dialyzed for 24 h to give the tagged IF₄₀₅ system. The IF₄₀₅ system was incubated in the presence of B₁₂, 1_{C5E} and 2_{C5E} conjugates. The Alexa Fluor 405 fluorophore was chosen because its excitation and emission profile does not overlap with the CypHer 5E dye. At least a 10-fold excess of IF was used throughout. The IF₄₀₅ incubated with B₁₂ shows uptake in the BeWo cells.

When the IF₄₀₅ was incubated with 1_{C5E} there was uptake and colocalization of the red and blue signals, confirming both IF and TT were present together. The blue signal commonly surrounds the red from the conjugate (FIG. 19C). This is consistent with multiple B₁₂ molecules being conjugated around the TT, which then are bound by IF₄₀₅. The 2_{C5E} conjugate also showed uptake and colocalization, but the enveloping effect of IF₄₀₅ noted for 1_{C5E} was greatly reduced and greater uptake was observed (FIG. 19D). This is consistent with the lower number of B₁₂s conjugated to the TT making up 2_{C5E}, thereby resulting in less IF₄₀₅ binding.

The 1C5E and 2C5E conjugates are both seen internalized in the cells as well. The fluorescence of these conjugates suggests a receptor mediated endocytosis uptake, due to the pH sensitive CypHer 5E dye. The TTC5E conjugate which shows no uptake confirms that the TT alone is not able to induce receptor mediated endocytosis. This suggests that the B₁₂ conjugation is required for the endocytosis of the conjugates. The colocalization of the IF405 with the conjugate systems further support the cubilin receptor mediated endocytosis. The work herein shows that it is possible for the uptake pathway to transport a 170 kDa conjugate system.

### REFERENCES FOR THE EXAMPLES

1. About kidney cancer. (2013, January 29). Retrieved from http://www.kidneycancer.org/knowledge/learn/about-kidney-cancer/2.
2. Bhat, S. (2010). Role of surgery in advanced/metastatic renal cell carcinoma. Indian Journal of Urology. 26(2), 167-176.
3. Motzer, R. J., Bacik, J., Schwartz, L. H., Reuter, V., Russo, P., Marion, S., and Mazumdar, M. (2004). Prognostic Factors for Survival in Previously Treated Patients With Metastatic Renal Cell Carcinoma. Journal of Clinical Oncology. 22(3), 454-463.
4. Griffin, N., Sohaib, S., and Martin Gore. (2007). Imaging in Metastatic Renal Cell Carcinoma. Oncologic Imaging. 189, 360-369.
5. Fass, L. (2008). Imaging and cancer: A review. Molecular oncology. (2), 115-152.
6. Randaccio, L., Geremia, S., Demitri, N., and Wuerges, J. (2010). Vitamin B₁₂: Unique Metalorganic Compounds and the Most Complex Vitamins. Molecules. (15), 3228-3259.
7. Kozyraki, R., Cases, O. (2013). Vitamin B12 absorption: Mammalian physiology and acquired and inherited disorders. Biochimie. (95), 1002-1007.
8. Waibel, R. (2008). New Derivatives of Vitamin B12 Show Preferential Targeting of Tumors. Cancer Research. (68), 2904-2911.
9. Furger E, Fedosov S. N., Launholt Lildballe D, Waibel R, Schibli R, Nexo, E., and Fischer, E. (2012) Comparison of Recombinant Human Haptocorrin Expressed in Human Embryonic Kidney Cells and Native Haptocorrin. PLoS ONE. 7(5): e37421.
10. Vortherms, A., Kahkoska, A., Rabideau, A., Zubieta, J., Andersen, L. L., Madsen, M., Doyle, R. (2011) A water soluble vitamin B12-Re(I) fluorescent conjugate for cell uptake screens: use in the confirmation of cubilin in the lung cancer line A549. Chem. Commun. (47), 9792-9794.
11. Kobayashi, H., Yoo, T., Kim, I., et al. (1996). L-Lysine Effectively Blocks Renal Uptake of ¹²⁵I- or ^{99m}Tc-labeled Anti-Tac Disulfide stabilized Fv Fragment. Cancer Res. (56), 3788-3795.
12. Henry, K., Balasingham, R., Vortherms, A., Platts, J., Valliant, J., Coogan, M., Zubieta, J., Doyle, R. (2013) Emission wavelength variation with changes in excitation in a Re(I)-bisthiazole ligand complex that breaks the Kasha-Vavliov rule. Chem. Sci., 2013, 4, 2490-2495.
13. Abram, U., Alberto, R. (2006). Technetium and Rhenium—Coordination Chemistry and Nuclear Medical Applications. J. Braz. Chem. Soc. (17) 1486-1500.

1. A pharmaceutical formulation for parenteral administration, the pharmaceutical formulation comprising intrinsic factor and B₁₂ or an analog thereof and a sterile pharmaceutically acceptable carrier for parenteral administration, wherein the B₁₂ or analog thereof is conjugated to a detectable label and/or therapeutic agent.

2. (canceled)

3. The composition of claim 1, wherein the intrinsic factor is bound to the B₁₂ or analog thereof.

4. The composition of claim 1, wherein the detectable label is a radionuclide.

5. The composition of claim 1, wherein the radionuclide is selected from the group consisting of copper-64, zirconium-89, yttrium-86, yttrium-90, technetium-99m, iodine-125, iodine-131, lutetium-177, rhenium-186 and rhenium-188.

6. The composition of claim 4, wherein the radionuclide is also a therapeutic agent.

7. The composition of claim 1, further comprising one or more pharmaceutically acceptable diluents, excipients, and/or carriers.

8. The composition of claim 1, wherein the location of conjugation of B₁₂ is selected from the group consisting of at the e-position, at the b-position, at the 5′ hydroxyl residue on the ribosyl group, and at the cobalt position.

9. The composition of claim 1 or 2, further comprising a linker.

10. A method of detecting a tumor in a subject, the method comprising:
a. administering to the subject a composition comprising intrinsic factor and B₁₂, wherein the B₁₂ is conjugated to a detectable label; and
b. detecting the detectable label to detect binding of the composition to cubilin in a subject, wherein the presence of the detectable label in a tissue that does not typically express cubilin indicates the presence of a tumor in the subject or the asymmetrical presence of the detectable label in a tissue comprising cells that are known to express cubilin indicates the presence of a tumor in the subject.

11. (canceled)

12. The method of claim 10, wherein the tumor comprises lung cancer.

13. The method of claim 10, wherein the tumor comprises renal cell carcinoma.

14. The method of claim 10, wherein the tumor expresses cubilin.

15. The method of claim 10, wherein the administering comprises intravenous administration.

16. The method of claim 10, wherein the detectable label is a radionuclide.

17. The method of claim 16, wherein the detecting comprises detecting the radionuclide label using positron emission tomography, single photon emission computed tomography, gamma camera imaging, or rectilinear scanning.

18. The method of claim 10, wherein free B₁₂ is administered prior to or concurrent with step (a).

19. The method of claim 10, wherein L-lysine is administered prior to or current with step (a).

20.-29. (canceled)

30. A method of delivering B₁₂ to a cell that expresses cubilin in a subject, the method comprising administering a complex of IF and B₁₂ to the subject intravenously.

31. The method of claim 30, wherein the cell is a cancer cell.

32. The method of claim 30, wherein the B₁₂ is conjugated to a detectable label and/or therapeutic agent.

33. A method of modulating cubilin function, the method comprising administering a complex of IF and B₁₂ to the subject intravenously.

34.-35. (canceled)
